(19) United States
(12) Patent Application Publication
(10) Pub. No.: US 2005/0153429 A1
(43) Pub. Date: Jul. 14, 2005

(54) Cell adhesion resisting surfaces

(76) Inventors: Andrea Liebmann-Vinson, Willowspring, NC (US); Richard P. Clarke, Raleigh, NC (US); Ruiling Xu, Cary, NC (US); Mohammad A. Heidaran, Cary, NC (US)

Correspondence Address: DAVID W. HIGHET, VP AND CHIEF IP COUNSEL;BECTON, DICKINSON AND COMPANY, 1 BECTON DRIVE, MC 110, FRANKLIN LAKES, NJ, 07417-1880, US

(21) Appl. No.: 10/994,879

(22) Filed: Nov. 22, 2004

Related U.S. Application Data
(62) Division of application No. 10/259,797, filed on Sep. 30, 2002.

(51) Int. Cl. G01N 33/53; B05D 3/00; C12M 1/34

(52) U.S. Cl. 435/287.2; 427/2.11

(57) ABSTRACT

A coated surface that resists cell adhesion comprises hyaluronic acid directly bound to a plasma-treated polymer surface. A process for producing the coated surface is disclosed as are further modifications of the hyaluronic acid by attaching ligand-binding polypeptides (antibodies or antibody binding proteins).

### BACKGROUND OF THE INVENTION

1. Field of the Invention

The invention relates to a coated surface that resists cell adhesion comprising hyaluronic or alginic acid directly bound to a plasma-treated polymer surface, and a process for producing the coated surface. The invention provides compositions, articles, objects, devices and methods comprising such a cell adhesion resistant (CAR) coated surface to which is bound a ligand binding polypeptide such as an antibody that binds selectively to a desired type of cell or molecule.

2. Background Information

The desirability of a surface that resists cell adhesion is well known in the art of tissue culture, inter alia, and considerable research has been carried out in the past to develop such surfaces. These are useful not only in tissue culture, but in other areas such as, for example, medical devices, where it is desirable to prevent bacterial cell adhesion and generally for surfaces that might otherwise be subject to fouling by the attachment of microorganisms. Hyaluronic acid (or hyaluronan), abbreviated HA, has been the subject of much investigation in this regard, and has been shown to produce surfaces with the desired resistant properties when immobilized thereon. One problem that has arisen, however, is that simply coating a surface with an HA layer of usually proves insufficient, as HA is water soluble, and dissolves in aqueous solutions over time. Thus, to achieve a wearable coating, it has generally been necessary to attach HA to a surface through stable chemical bonding.

U.S. Pat. No. 6,129,956 to Morra discloses that HA and alginic acid (AA) can be covalently coupled and immobilized onto polystyrene using polyethyleneimine (PEI) or poly-L-lysine (PLL) as an intermediate coupling layer, or can be covalently linked with a bi-functional alkoxy silane coupling agent which couples HA to a plasma-treated surface. Both of these methods entail a considerable investment of time and effort.

Mason et al., Biomaterials 21 (2000) 31-36 disclosed immobilization of HA to polymers treated with ammonia plasma, providing that a particular coupling agent was employed. Thus, after ammonia plasma treatment of a surface, HA could not be successfully coupled to either polystyrene (PS), polypropylene (PP), or polytetrafluoroethylene (PTFE) using ethyldimethyl-aminopropyl carbodiimide (EDC) as a condensing agent. Only when HA was modified using adipic dihydrazide was HA successfully coupled by EDC to these treated polymers. Thus, according to Mason et al., HA is coupled to surface-bound —NH₂ groups only in the presence of a “spacer arm” (the adipic dihydrazide) between the surface amine and the HA carboxyl group. However, modifications of HA are undesirable and azide compounds in general pose safety concerns.

Therefore, there remains a need for a simplified method of preparing a surface that resists cell adhesion.

Many reports describe coupling active biological materials to solid supports. For example, covalent attachment of antibodies to PS dishes and use of these derivatized dishes for cell depletion procedures in a panning process (Larsson, P H. et al., J Immunol Meth (1989) 116:293-298). Derivatization of a PS surface through covalent linkage to antibodies or their fragments, for use in immunoassays, etc., is described by Peterman, J H, et al., J Immunol Meth (1988) 111:271-275 and Chu, V P et al., J App Polymer Sci (1987) 34:1917-1924.

A number of patents assigned to Applied Immune Sciences, Inc., and scientific publications of members of this company, disclose covalent derivatization of PS surfaces and their use to covalently immobilize ligand binding proteins, primarily antibodies, and used these to capture (positive selection) and remove (negative selection) certain subsets of cells from mixed cell populations. See, for example, Okrongly (U.S. Pat. Nos. 4,933,410 and 5,283,034), Clark (U.S. Pat. Nos. 4,978,724 and 5,241,012(, Lebkowski, JS et al., In: Recktenwald, D et al., eds., Cell Separation Methods and Applications, Marcel Dekker, Inc., New York, 1998, pp. 61-85; Okarma T et al., Prog Clin Biol Res, 1992, 377:487-504; A. E. Berson et al., Biotechniques, 1996 20:1098-103). Devices (PS tissue culture flasks) prepared according to Okrongly or Clark were used for negative depletion and positive selection of specific murine cell populations. In some cases, the selecting antibodies were bound noncovalently to mouse anti-rat antibody which were bonded covalently to the surface. Also disclosed were covalently immobilized lectins with specificity for certain saccharides expressed differentially on cells. These methods were used in single or multiple steps to enrich functional hematopoietic progenitor cells from bone marrow (Schain, L R et al., J Hematother, 1994, 3:37-46; Cardoso A A et al., J Hematother 1993, 2:275-9).

However, none of the foregoing references directed to coupling of ligand binding proteins disclose use of cell-adhesion resistant surfaces to which such proteins are immobilized nor do they suggest any reason to make or used such a surfaces. Therefore, it was unexpected to find, as described herein, that a surface designed to resist adherence/binding of proteins, cells, etc. could be made selectively attractive to predetermined molecules, cell surface structures, etc., without losing its general resistive properties by attaching specific ligand binding molecules to that resistive surface.

### SUMMARY OF THE INVENTION

It has been unexpectedly found that HA can be directly bound to a plasma-treated surface, without the need for a coupling agent and without treating the surface with PEI, PLL, poly-D-lysine (PDL) or another polycationic substance.

Therefore, it is an article of the invention to provide a method to covalently immobilize HA onto a surface to obtain surfaces that resist cell adhesion. In particular, the present invention provides a method of obtaining a nitrogen-containing surface directly on polystyrene and other polymeric surfaces by treatment with plasma, and subsequently immobilizing HA thereon, without requiring the use of an intermediate binding layer or the use of chemical coupling agents.

In another embodiment, the present invention provides a method of providing directly immobilized HA on (polymeric) nitrogen-containing surfaces, without requiring the use of an intermediate binding layer such as polyethyleneimine or the use of chemical coupling agents.

Examples of such surfaces are ammonia plasma-treated polymers and Primaria™-treated polystyrene surfaces. Polymeric substrates suitable for use in the invention include polystyrene, polypropylene, polyethylene terephthalate, polylactide, cellulose and the like.

In yet another embodiment, the present invention provides a surface that resists cell adhesion. The surface is comprised of a layer of hyaluronic acid that is directly bound to a polymer, such as polystyrene, through an amine group, and does not contain an intermediate binding layer or linker group such as PLL.

Surfaces formed according to the method of the invention will be useful for the same purposes as HA- or alginate-coated and other cell adhesion resistant surfaces that were previously known in the art. When not further treated, such surfaces will be useful for resisting cellular adhesion and growth. They can also be further treated by means known in the art to selectively attach additional agents having specific desired properties. It may be advantageous in some cases to couple biologically active materials that have specific affinities for target cells or compounds.

This invention is further directed to a method of immobilizing ligand binding polypeptides (LBPs), preferably antibodies, to a modified solid surface as described above that prevents non-specific cell and protein adsorption. This is achieved by covalently bonding an LBP such as (a) an antibody to a cellular target, or (b) a capture protein, such as Staphylococcal protein A (SpA) and Streptococcal protein G (SpG), that naturally binds to immunoglobulin (Ig) molecules, or other proteins that can be made to interact with antibodies in a specific manner. Examples of the latter “capture” proteins are avidin or streptavidin which bind with exceedingly high affinity to biotin that is chemically conjugated to a soluble target-specific antibody.

In this embodiment, the surface is first modified as described above to create a CAR surface. Once HA, for example, is bonded to the surface, free hydroxyl groups of the HA are oxidized to aldehydes, for example with a periodate (e.g., NaIO₄). Polypeptide can now react with these aldehyde groups through their free primary amine groups (N-terminus, Lys or Arg side chains, etc.). In the presence of a suitable reducing agent, e.g., a borohydride such as cyanoborohydride, reductive amination takes place resulting in covalent bonding between the polypeptide and the reactive aldehydes on the saccharide rings of the HA (or AA).

Similarly, the COO⁻ groups of the CAR material, preferably HA or AA, that is bonded to the surface may be activated to form reactive intermediate esters (o-acylisourea) by the addition of ethyldimethylaminopropyl-carbodiimide (EDC). This intermediate is highly unstable and subject to hydrolysis, leading to the cleaving off of the activated ester intermediate, forming an isourea, and regenerating the —COO⁻ group. To stabilize this unstable reactive intermediate and increase reaction yield, N-hydroxysulfosuccinimide (sulfo-NHS) or an equivalent reactive intermediate stabilizing agent is added to the reaction. Free amino groups of a peptide or polypeptide (N-terminus, Lys and Arg side chains, etc.) can now react with these reactive intermediate esters or the stabilized reactive intermediate esters, forming a stable amide bond. This results in covalent bonding between the peptide or polypeptide and the reactive ester on the saccharide rings of the HA or AA.

The present invention provides A method for producing a cell-adhesion resistive (CAR) solid phase surface to which is covalently bonded at least a first ligand binding polypeptide, comprising the steps of:
(a) coating a polymer surface with HA, AA, or derivative in accordance with claim 1;
(b) oxidizing the HA, AA or derivative to create an amine-reactive group; and
(c) exposing the oxidized HA, AA or derivative to a first ligand-binding polypeptide wherein covalent bonds are formed between amino groups of the polypeptide and the amine-reactive group, resulting in the covalent bonding of the polypeptide to the HA, AA or derivative,
   thereby producing the CAR surface to which is covalently bonded first ligand-binding polypeptide.

A preferred surface is selected from the group consisting of polystyrene, polyethylene, polypropylene, polyethylene terephthalate, polytetrafluoroethylene, polylactide and cellulose.

In the above method
(i) the oxidizing step (b) may be performed by providing an oxidizing agent, preferably periodate, that generates reactive aldehyde groups on the HA, AA or derivative, and
(ii) step (c) additionally comprises providing a reducing agent to the polypeptide and the surface that effects reductive amination that results in the covalent bond formation between the amino groups of the polypeptide and the reactive aldehyde groups.

Also provided is the above method, wherein step (b), either before step (c) or contemporaneously therewith, also comprises the step of converting carboxylate groups of the HA, AA or derivative to reactive esters by exposure to a carbodiimide and a reactive intermediate ester stabilizing compound.

The reactive intermediate ester stabilizing compound in the above method is selected from the group consisting of N-hydroxysuccinimide, hydroxysulfosuccinimide and hydroxybenzotriazolohydrate.

The above method may comprise, after step (c),
(d) contacting the covalently bonded first ligand binding polypeptide with a second ligand binding polypeptide that is a ligand for the first ligand binding polypeptide under conditions that result in the noncovalent binding of the second polypeptide to the first polypeptide.

Preferred first ligand-binding polypeptides are (a) an antibody, (b) a receptor, (c) an immunoglobulin binding protein, (d) avidin or streptavidin, (e) a lectin, (f) a cell adhesion molecule or (f) an extracellular matrix protein or (g) a synthetic peptide. An immunoglobulin-binding protein may be selected from the group consisting of a native or recombinant staphylococcal protein A, a native or recombinant staphylococcal protein G, and recombinant protein A/G. The second ligand-binding binding polypeptide may be (a) an antibody or antigen-binding fragment thereof, (b) a receptor, (c) a lectin, (d) a cell adhesion molecule or (e) an extracellular matrix protein or (f) a synthetic peptide.

In one embodiment, (a) the first ligand-binding polypeptide is protein A, protein G, or recombinant protein A/G; and (b) the second ligand binding polypeptide is an antibody or antigen-binding fragment thereof. In another preferred embodiment, (a) the first ligand-binding polypeptide is avidin or streptavidin; and (b) the second ligand binding polypeptide is a biotinylated antibody.

The foregoing methods are advantageously used to immobilize a desired LBP which acts as a capture agent. A preferred LBP is an antibody of desired specificity that binds, for example, to cells that are being isolated, enriched or depleted. One articleive is to use this immobilized antibody to positively select cells which express on their surface an epitope or antigen for which the antibody is specific. Conversely, these surfaces can be used for negative selection as well. A preferred antibody for immobilization and use in accordance with this invention is anti-CD34 since the CD34 marker is expressed on early hematopoietic stem and progenitor cells that, when isolated, have many beneficial uses.

Using anti-CD34 antibodies as an exemplary antibodies or LBPs, the following embodiments are provided. SpA or SpG is immobilized to an HA- or AA-treated polymeric surface, and then used to immobilize (noncovalently) anti-CD34 mAbs. In another embodiment, avidin or streptavidin is bonded to the HA or AA surface and used to immobilize (noncovalently) biotinylated anti-CD34 antibodies. In another embodiment, the anti-CD34 antibody is directly coupled (covalently) to an HA- or AA-treated polymeric surface as described herein. This results in a capture surface that is coated with a desired antibody but at the same time resists nonspecific binding of cells that do not express CD34.

A capture surface with specific patterns of capture agent, preferably antibody, can be created wherein regions of bound antibody are separated by regions that lack antibody but that display the cell adhesion resisting substance. Such a patterning permits development of specialized antibody capture arrays.

Also provided is an article made by any of the above methods that comprises a CAR material bonded to a solid surface, and, in contact, preferably bonded to, the CAR material is a first LBP or a first LBP binding a second LBP

These and other embodiments of the invention are described in further detail and specific examples set forth below.

### BRIEF DESCRIPTION OF THE DRAWINGS

FIG. 1 shows the O/C and N/C ratios for each plasma treatment condition (A-E) described in Example 5.

FIG. 2 shows MC3T3 cells fixed and stained with hematoxylin in plasma-treated and HA coated dishes as described in Example 7. Shading indicates the presence of cells. The three plasma control dishes (top row) are stained [bright purple], indicating the presence of a cell layer. No staining was found in the plasma treated and HA coated dishes for Conditions A and B, indicating that no cells attached to these surfaces. Little cell attachment is found in the two plasma treated and HA coated dishes for condition E, possibly due to minor defects in the coating. [This may also be transformed to a B&W bimage - the bright purple is then grey and the surfaces with no cells are white]

FIGS. 3 is a schematic representation of the CAR surface to which is immobilized an exemplary LBP, anti-CD34 mAb, showing the different “layers” built up on the polystyrene surface.

FIG. 4 is a schematic illustration representing the steps in two different processes of modifying a CAR surface to immobilize a LBP (either a single LBP or both a primary (1°) LBP and a secondary (2°) LBP. In this embodiment, the layer of CAR material is exemplified as HA or AA which may be bonded directly to a PS surface or bonded to an intermediate layer (here, exemplified by PEI) which is directly bonded to the PS surface.

FIGS. 5-7 show direct fluorescence measurements of antibody binding to surfaces of the invention using a BMG fluorometer. 96 well microplates coated with HA were treated to create three different types of surfaces for immobilizing a murine mAb. The surfaces were evaluated for maximal binding of anti-CD34 mAb. Surface 1 (FIG. 5) was modified with SpG. Surface 2 (FIG. 6) was modified with SpA. Surface 3 (FIG. 7) was modified with avidin (and tested with biotinylated anti-CD34 mAb. Efficiency of coupling of the mAb to each surface was measured fluorimetrically using a fluorescent 2° antibody (anti-Ig) to which was coupled to the fluorescent dye Alexa 488® (see Examples). Representative results are shown.

### DETAILED DESCRIPTION OF THE INVENTION

In describing preferred embodiments of the present invention, specific terminology is employed for the sake of clarity. However, the invention is not intended to be limited to the specific terminology so selected. It is to be understood that each specific element includes all technical equivalents, which operate in a similar manner to accomplish a similar purpose. Each reference cited here is incorporated by reference as if each were individually incorporated by reference.

Abbreviations:
CAR: cell adhesion resisting (or resistant or resistive).
ESCA: Electron spectroscopy for chemical analysis
EDC: 1-ethyl-3-(3-dimethylaminopropyl)-carbodiimide
MES: 2-[N-Morpholino]ethane sulfonic acid
NHS: N-hydroxysuccinimide
Sulfo-NHS: N-hydroxysulfosuccinimide
PEI: polyethyleneimine
PLL: poly-L-lysine
PP: polypropylene
PS: polystyrene
PE: polyethylene
PET: polyethylene terephthalate
PTFE: polytetrafluoroethylene
LBP: ligand-binding polypeptide(s)

All solutions are w/v in deionized H₂O, unless otherwise indicated.

As used herein, the term “polymer” or “polymeric substrate” is intended to refer to the composition of the article or surface which is to be plasma treated, and on which the HA surface according to the invention will be coated.

PEI, PLL and other coatings used in the prior art to facilitate HA binding, although they may also be polymeric in character, will generally be referred to as linking or coupling agents or layers, intermediate binding layers and/or by other specific functional terms.

Coupling Reaction Between Carboxyl Groups on HA and Amine Groups on a Surface

HA is an anionic polysaccharide composed of repeating units of β-1,4-glucuronate-β-1,3-N-acetylglucosamine. A reactive —CO₂ group is present on every repeat unit of HA that can be utilized to covalently couple HA to an amine containing surface using the methods of the invention. Ethyldimethylaminopropyl-carbodiimide (EDC) reacts with —COOH to create an active-ester (o-Acylisourea) intermediate. This intermediate is highly unstable and subject to hydrolysis, leading to cleaving off the activated ester intermediate, forming an isourea, and regenerating the —COOH group. To stabilize this unstable reactive intermediate and increase reaction yield, sulfo-NHS or another equivalent agent is added to the reaction.

HA that has been covalently immobilized by the methods of the present invention has been demonstrated to prevent cell adhesion, e.g. the attachment of murine calvaria-derived osteoblast cells (MC3T3 cells). Furthermore, the surfaces prepared by the present methods were resistant to peel off after extended times in culture.

HA immobilized directly on plasma-treated surfaces has the advantage that no intermediate polymer layer (e.g. polyethyleneimine, poly-D-Lysine, or poly-L-lysine) or other “spacer” moiety is needed. A layer of HA can be directly immobilized onto the PS surface without losing its cell-adhesion preventing characteristics. The present invention thus avoids the necessity of additional steps of using of intermediate polymer layers, e.g. PEI or polylysine, or spacer groups.

The invention can be used, for example, for coating of tissue culture ware to prevent cell adhesion and growth, for creating surfaces for further modification with biologically relevant ligands (e.g. peptides, ECMs, proteins), for non-fouling surfaces to prevent bacterial cell adhesion, and surfaces for proximity scintillation and fluorescence polarization assays. As described in more detail below, the same tissue culture devices may have immobilized thereon specific ligand binding polypeptides, preferably antibodies or antibody-binding polypeptides.

The use of plasma techniques are familiar to those of skill in the art (see, for example, Garbassi F. et al., “Polymer Surfaces, from Physics to Technology”, Wiley, Chichester, 6, 1994, and N. Inagaki “Plasma Surface Modification and Plasma Polymerization, Technomic Publishing Company, Lancaster, 1996). In the present invention, the plasma treatment process may be any process that is capable of causing nitrogen to be incorporated onto the surface of the article resulting in reactive amine or other nitrogen-containing groups, including direct as well as remote plasma treatment methods. Examples of suitable plasma treatments are ones using reactive gases such as nitrogen, nitrogen oxide, nitrogen dioxide or ammonia in the gas phase, alone or in mixture with air, argon or other inert gases and may be preceded or followed by treatments employing argon or other inert gases. The plasma may be sustained over the full treatment time or may be administered in pulses. Preferably, the plasma gas is ammonia, and treatment is performed with a power charge of between 1 and 400 W, preferably between 10 and 150 W, a pressure between 10 mtorr and 10 torr, and a treatment time between 1 second and 1 hour, preferably between 10 seconds and 30 minutes.

Plasma-treated polystyrene can be prepared, for example by pumping the treatment chamber to a 0.3 mTorr base pressure, establishing a 200 mTorr argon atmosphere, and applying a 60 sec argon plasma treatment, followed by a 120 sec, 375 mTorr NH3 plasma treatment at 95 W. Other suitable treatments will be known to those of skill in the art, and examples are set forth below.

The purpose of the plasma treatment is to create a high surface concentration of covalently attached amine groups. The surface can then be reacted with hyaluronic acid or a derivative thereof, or alginic acid (alginate), in the presence of a condensing agent such as EDC, in aqueous solution or dicyclohexylcarbodiimide (DCC), in organic solvents. For optimal results, a molecule able to enhance the reaction promoted by EDC, such as N-hydroxy-succinimide (NHS), hydroxy-sulfosuccinimide or hydroxybenzotriazolo hydrate should also be present. Although the success of the invention is not intended to be bound to a particular theory, attachment of HA to the amine containing surface is believed to occur through a mechanism wherein (for example) EDC and NHS combine to create an active ester polysaccharide with a carboxyl group capable of coupling to an amine. When coupling occurs, NHS is released. Other compounds known in the art that are able to react with EDC in this manner and which serve as reactive intermediate ester stabilizing compounds should also be effective in the invention.

Other plasma treatment methods for producing surfaces with amine and other nitrogen-containing groups are also suitable, and are known to those of skill in the art. Following plasma treatment of the surface to be coated, the plasma treated surface is exposed to an aqueous solution containing HA or a derivative thereof, or AA in the presence of a carbodiimide, preferably EDC. The term “expose” or “exposing” as used herein is intended to include any type of contact made between a liquid and a solid, for example by pipetting, pouring, spraying, dripping, immersing, pouring, dipping, injecting, etc., without limitation.

A reactive intermediate ester stabilizing compound that substantially increases the coupling yield by stabilizing the reactive intermediate formed by the carbodiimide is also present. Such compounds are generally selected from the class of N-hydroxysuccinimides and aryl or heterocyclic derivatives thereof. Preferred N-hydroxysuccinimides include, but are not limited to, N-hydroxy-succinimide (NHS), hydroxy-sulfosuccinimide (sulfo-NHS), hydroxy-benzotriazolo hydrate.

Suitable derivatives of HA that may be used in the invention will be known to the skilled artisan, and are described, for example, in U.S. Pat. No. 4,851,521. These include partial esters of hyaluronic acid with alcohols of the aliphatic, araliphatic, cycloaliphatic and heterocyclic series and salts of such partial esters with inorganic or organic bases. Similar derivatives of alginic acid should also be useful.

Surfaces prepared according to the method of the invention are very effective in resisting adhesion of cells, as shown in the examples herein below, and can be prepared much more economically and efficiently than those requiring an intermediate layer of a compound comprising nitrogen-containing groups, such as PEI, PLL or PDL.

Ligand-binding Polypeptides (“LBP”)

As used herein, an LBP is any polypeptide that has affinity for, and, under suitable conditions, binds to, a binding partner or ligand, also referred to herein as a “target.” A preferred LBP is one which binds to a target that is on a cell surface, whether it be a protein, a carbohydrate, a lipid, or any structure comprising a combination of these basic biochemical building blocks, such as a glycoprotein or glycopeptide, glycolipid or proteolipid.

Useful LBP's may be naturally occurring polypeptides that are obtainable by direct isolation (or by genetic engineering) in their native structural form. Examples are polypeptide receptors for hormones such as insulin receptors, glucagon receptors, receptors for proteinaceous endocrine hormones, receptors for neuropeptides, or receptors for cytokines, Ig molecules (e.g., bacterial Ig binding molecules, Fc receptors, anti-Ig antibodies), complement components, inflammatory peptides, plant lectins (such as soybean agglutinin, wheat germ agglutinin, phytohemagglutinin, concanavalin A, and the like, set forth in more detail below). Other LBPs are cell adhesion molecules that bind to either the same (homotypic) or different (heterotypic) cell adhesion molecules.

The only requirement for a useful LBP in the present compositions and methods is that it bind to its ligand with specificity and sufficient affinity when in immobilized form, to permit binding of cells (or other molecules) for purposes such as those disclosed herein.

The most preferred LBP of the present invention is an antibody, preferably a monoclonal antibody (mAb).

Standard references for antibodies and related immunological aspects of the present invention, which are hereby incorporated by reference in their entirety, include: A. K. Abbas et al.,

Cellular and Molecular Immunology (Fourth Ed.), W. B. Saunders Co., Philadelphia, 2000, C. A. Janeway et al., Immunobiology. The Immune System in Health and Disease, Fifth ed., Garland Publishing Co., New York, 2002 Harlow, E, and Lane, D. Using Antibodies: A Laboratory Manual. New York: Cold Spring Harbor Laboratory Press, 1998. Howard, G C and Bethell, D R, Basic Methods in Antibody Production and Characterization, CRC Press, Boca Raton, 2001. For analysis of immobilized antibodies, see, for example, Butler, J E, The Behavior of Antigens and Antibodies Immobilized on a Solid Phase (Chapter 11) In: STRUCTURE OF ANTIGENS, Vol. 1 (Van Regenmortel, M, ed), CRC Press, Boca Raton 1992, pp. 209-259. 100571 The present invention utilizes antibodies, both polyclonal and monoclonal, as LBP's. The antibodies may be xenogeneic, allogeneic, syngeneic (relative to the species of cells being bound), or modified forms thereof, such as humanized or chimeric antibodies (see below). The term “antibody” is also meant to include both intact molecules as well as antigen-binding fragments thereof, such as Fab and F(ab′)₂ fragments which lack the Fc fragment of an intact antibody. Also included are Fv fragments (Hochman, J. et al. (1973) Biochemistry 12:1130-1135; Sharon, J. et al.(1976) Biochemistry 15:1591-1594).). These various fragments are produced using conventional techniques such as protease cleavage or chemical cleavage (see, e.g., Rousseaux et al., Meth. Enzymol., 121:663-69 (1986))

Polyclonal antibodies are obtained as sera from immunized animals such as rabbits, goats, rodents, etc. and may be used directly without further treatment or may be subjected to conventional enrichment or purification methods such as ammonium sulfate precipitation, ion exchange chromatography, and affinity chromatography (H. Zola et al., in Monoclonal Hybridoma Antibodies: Techniques and Applications, CRC Press, 1982)). The mAbs may be produced using conventional hybridoma technology, such as the procedures introduced by Kohler and Milstein (Nature, 256:495-97 (1975)),-and modifications thereof (see above general immunology references). Commercially available mAbs are preferred.

The antibody may be produced as a single chain antibody or scFv instead of the normal multimeric structure. Single chain antibodies include the hypervariable regions from an Ig of interest and recreate the antigen binding site of the native Ig while being a fraction of the size of the intact Ig (Skerra, A. et al. (1988) Science, 240: 1038-1041; Pluckthun, A. et al. (1989) Methods Enzymol. 178: 497-515; Winter, G. et al. (1991) Nature, 349: 293-299); Bird et al., (1988) Science 242:423; Huston et al. (1988) Proc. Natl. Acad. Sci. USA 85:5879; Jost C R et al,. J Biol Chem. 1994 269:26267-26273; U.S. Pat. No. 4,704,692, 4,853,871, 4,94,6778, 5,260,203, 5,455,030.

A hybrid or chimeric antibody which can be prepared by genetic engineering (see, e.g., Cabilly et al., U.S. Pat. Nos. 4,816,567 and 6,331,415; Morrison et al., U.S. Pat. No. 5,807,715) or by protein manipulation after the antibody has been synthesized. Biochemical method for constructing such a hybrid Ab1-Ab2 antibody and hybridoma-based recombinant methods for the same are disclosed, in for example, Hillyard et al., U.S. Pat. No. 5,413,913 and references cited therein. A hybrid or chimeric antibody of the present invention thus comprises two “half-molecules,” one with specificity of mAb1 and the other with specificity of mAb2. Such a hybrid antibody has advantages over a tail-to-tail conjugate as taught in the prior art, which is formed by a bifunctional coupling agent. The advantages include ease of preparation, the preservation of the correct stoichiometry and stereochemistry of both antibodies and the retention of the binding affinity of each fragment.

LBP Fragments and Engineered LBPs

An LBP is also intended to include the ligand-binding fragment, domain or portion of a full-length polypeptide. To illustrate, if the complete or native LBP is a cell-surface receptor protein, the most useful LBP fragment may be the extracellular domain (ECD) of the receptor. In the case of an antibody, the LBP fragment may be any antigen-binding fragment such as an F(ab′)₂, Fab or Fv fragment. Modified, engineered forms of a native LBP such as single chain antibody (scFv; Skerra, A. et al. (1988) Science, 240: 1038-1041; Pluckthun, A. et al. (1989) Methods EnzymoL 178: 497-515; Winter, G. et al. (1991) Nature, 349: 293-299); Bird et al., (1988) Science 242:423; Huston et al. (1988) Proc. Natl. Acad. Sci. USA 85:5879; Jost CR et al,. J Biol Chem. 1994 269:26267-26273; and U.S. Pat. Nos. 4,704,692, 4,853,871, 4,946,778, 5,260,203 and 5,455,030), a chimeric antibody (Cabilly et al., U.S. Pat. Nos. 4,816,567 and 6,331,415), a CDR-grafted antibody, and the like are within the scope of this invention.

Double (Sandwiched) LBPs

In one embodiment of this invention, the LBP that is intended to bind to the target structure is directly immobilized to the solid phase by covalent bonding to the CAR material, preferably HA. A preferred example of a directly immobilized LBP is an antibody. However, in other embodiments, increased efficiency of target (commonly, cell) capture is achieved by having a first LBP covalently bonded directly to the solid phase, to the HA or other CAR coating layer and, bound non-covalently to the first LBP is a second LBP which is both a ligand for the first LBP and a binding partner for the intended target. In one embodiment, the first LBP is an antibody-binding polypeptide which can “capture” an antibody without impeding its ability to recognize and bind to the ultimate target. Examples of useful antibody-binding polypeptides are certain bacterial proteins such as staphylococcal protein A (SpA) and protein G (SpG) (see below)) which have the native capacity to bind certain Ig molecules, usually at the Fc portion distal from the Ig's antigen-binding site. A polypeptide can be engineered to function as an antibody-binding polypeptide. A preferred examples is streptavidin or avidin, which bind naturally with extremely high affinity to biotin. Thus, if streptavidin is directly bonded covalently to the solid surface via its reaction with HA (first LBP), it can bind a biotin-conjugated antibody (second LBP) non-covalently but with very high affinity. This antibody is then used to bind to the target, and, for example, to capture cells.

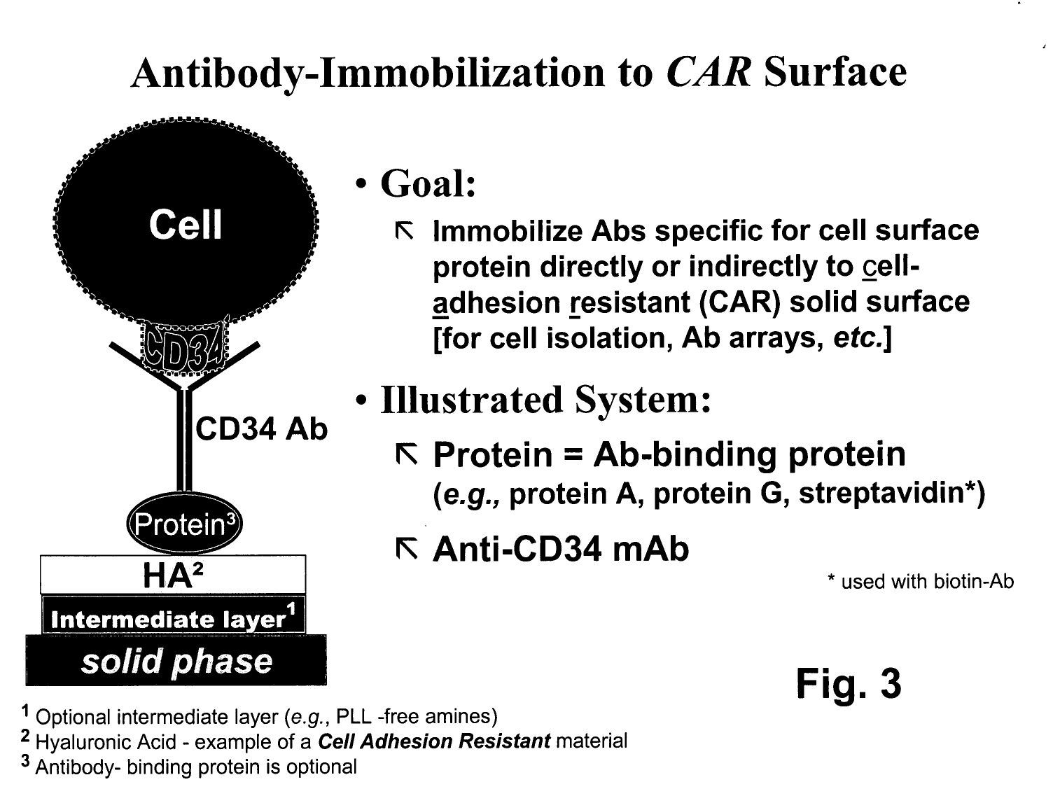

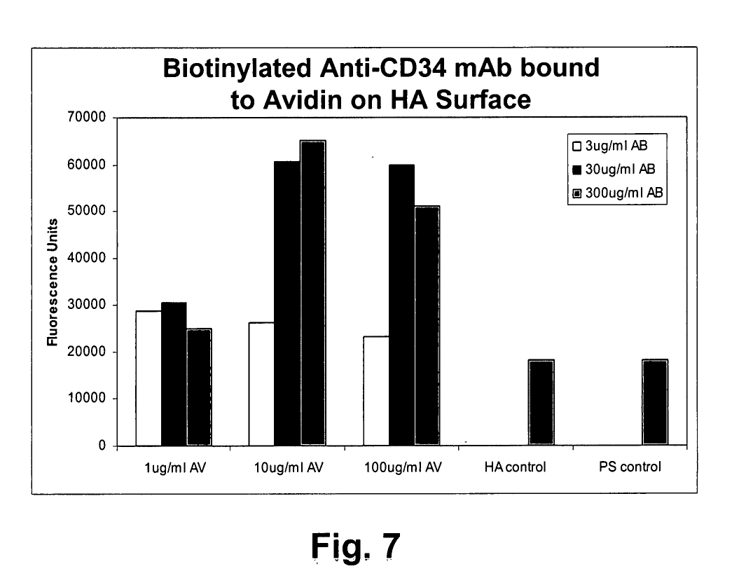

An antibody or fragment thereof serving as the ultimate LBP, whether as a first LBP or as a second LBP, may be specific for any epitope of interest which is expressed on a cell surface and can be employed as a means to capture cells expressing that epitope. Such cell surface epitopes, many of which are known “markers” for cells of certain differentiation lineages antigens, are well-known in the art and need not be described herein. Similarly, mAbs specific for such known cell surface markers, are well-known in the art. Many are available commercially. As an example, if the cells to be captured by the immobilized antibodies and devices of the present invention are hematopoietic stem cells, then a stem cell markers should be selected. CD34 is a known antigen present on early hematopoietic stem cells, and anti-CD34 mAbs are also well known and commercially available. As illustrated in FIGS. 3-7, the methods of the present invention are used to immobilize anti-CD34 mAbs on a CAR. Such a composition is used for efficient capture and isolation/enrichment of human CD34+ stem cells without the undesirable nonspecific adherence of cell to the surface which would occur in the absence of the CAR substance, exemplified as HA, comprising a layer of the solid phase to which the anti-CD34 mAbs are bound.

Immunoglobulin Binding Polypeptides as LBP

As noted above, in a preferred embodiment, the present invention is directed to compositions, devices and methods in which an Ig-binding protein is immobilized covalently to a CAR solid surface, where it further immobilizes antibodies (noncovalently). These latter antibodies then are able bind to target structures to perform the ultimate objectives of the invention.

Preferred examples of Ig-binding proteins are SpA, SpG, a recombinant chimeric fusion protein “protein A/protein G” (pA/G), and proteins from other sources such as mannose-binding lectin (MBL; previously known as mannan-binding protein or MBP) and jacalin (from plants).

SpA is a highly stable surface receptor produced by Staphylococcus aureus, which is capable of binding the Fc portion of Ig molecules, especially the Fc of IgGs, from a large number of species (Boyle, MDP et al., “Bacterial Fc Receptors.” Biotechnology 5:697-703 (1987); Boyle, MPD., ed. Bacterial Immunoglobulin-Binding Proteins, Microbiology, Chemistry and Biology, Vol I. Academic Press, San Diego (1990)). See Table 1, below. SpA has a molecular mass of about 42 kDa (based on sedimentation data; Bjork et al., 1972 Bjork, I et al., Eur J Biochem. 29:579-584 (1972) although SpA runs anomalously slowly on SDS polyacrylamide gels (at an apparent molecular weight of 55-56 kd; ibid.). SpA is monomeric and lacks Cys residues. It has a pI of 4.85-5.10 and is stable at pH 1.0-12.0. One SpA molecule can bind at least 2 molecules of IgG simultaneously (Sjoquist, J et al., Eur J Biochem 29:572-578 (1972)). SpA has been immobilized onto a solid support to facilitate the purification and recovery of either polyclonal or monoclonal immunoglobulins. Immobilized SpA has been used for extracorporeal immunoadsorption in the treatment of various diseases (Jia L et al., Biomed Chromatogr. , 1999, 13:472-7; Murphy RM et al., Mol Biother., 1989, 1:186-207; Watt RM et al., Transfus Sci., 1992, 13:233-53; Hakansson L et al., Eur J Cancer Clin Oncol., 1984, 20:1377-88; Korec S et al., J Biol Response Mod., 1984, 3:330-5; Terman DS., Int J Artif Organs., 1982, 5:77-80) and even used in vivo to treat a chemotherapy-induced hemolytic-uremic syndrome which is mediated by antibodies (Watson, PR et al., Cancer 64:1400-1403 (1989).

Recombinant SpG (Fahnestock, S R et al., J. Bacteriology 167:870 877. (1986); Trends Biotechnol 5:79 84 (1987)) is a highly stable surface receptor from Streptococcus sp. Lancefield Group G, produced in Escherichia coli, which is capable of binding the Fc portion of Ig's especially IgGs, from a large number of species (Boyle et al., 1987, supra). See Table 1. SpG has a molecular mass of 22.6 kDa, though its apparent MW by SDS PAGE is 32 kDa. SpG is a monomer lacking Cys residues. Its pI is 4.5 and it is stable at pH: 2-10 and at 80° C. (for 10 min at pH 7). Each protein G molecule can bind 2 molecules of IgG, allowing the formation of a precipitate. SpG has been immobilized onto a solid support to facilitate the purification and recovery of either polyclonal or monoclonal immunoglobulins.

SpA, from Staphylococcus aureus, and SpG, from Streptococcus sp. (Lancefield Group G), both exhibit an affinity for the constant region (Fc) of a diverse array of immunoglobulins (Ig) from many species. The specificities for these Fc-binding proteins differ, although there is some overlap in the Ig class, subclass and species range (Boyle et al., 1987,supra; Boyle, 1990, supra). In order to produce a single protein with an expanded species and subclass range of Fc-binding activity, the genes encoding the Fc-binding domains of both SpA and SpG were fused. In this construction, the SpG gene sequences encoding the serum albumin binding site and the membrane anchor region were excluded.

The relatively new Ig Fc-binding protein, pA/G, is synthesized as a fusion protein having a molecular weight of 50 kDa and a statistically determined pI of 6.9. pA/G is a recombinant protein derived from a hybrid gene composed of the Ig-binding domains of the Staphylococcus aureus protein A gene (including domains E, D, A, B and C), and the Ig-binding domains of the Streptococcus protein G gene (C2 and C3). It is expressed in Escherichia coli and affinity purified. The fusion gene product contains 455 amino acid residues (41 lysines, no cysteines) and seven Fc-binding domains (5 from protein A, 2 from protein G). pA/G fusions exhibit a sensitivity for Ig comparable to that exhibited by SpA and SpG (See Tables), and, with respect to human Ig, show higher or equal avidity in comparison to the best of the parental proteins. (Eliasson, M et al., J. Biol. Chem. 263:4323-4327 (1988); Eliasson, M et al., J. Immunol. 142:575-581 (1989)). They also exhibit a broader specificity than either SpA or SpG alone. Sun, S and Lew A M (J. Immunol. Meth. 152:43-48 (1992)) reported that a pA/G fusion bound Ig from most mammalian species, including primates, camivora, artiodactyla, perissodactyla, lagomorpha, and rodentia; but did not bind proboscidea, marsupialia or avia. Unlike native SpG, pA/G does not bind albumin from human nor mouse serum. For these reasons pA/G has been considered a more versatile and convenient reagent for certain immunological techniques than either protein A or protein G alone. pA/G binds the Fc portion of all human IgG subclasses, IgA, IgE, and IgM, and mouse IgG subclasses 1, 2a, 2b, and 3. In addition, it binds IgGs from other species including monkey, rabbit, pig, guinea pig, cow, dog, cat, goat, horse and sheep. It will not bind well to rat Ig, chicken Ig, mouse IgA or mouse IgM. pA/G will not bind bovine, murine or human serum albumin. pA/G may be used wherever SpA or SpG are known to be useful.

Two Ig-binding lectins are described here (separately from the section below devoted specifically to lectins). Jacalin is a lectin present in the seeds of the Jackfruit, Artocarpus integrifolia. Jacalin has a molecular weight of approximately 50 kDa and is composed of four subunits, two 10 kDa and two 16kDa subunits. Jacalin binds galactose (Gal) and in glycoproteins, appears to bind only O-glycosidically linked oligosaccharides, preferring the structure Gal(β1,3)GalNAc, to which it binds in a mono- or disialylated form. Jacalin specifically binds human secretory IgA and can be used to separate human IgA from other serum glycoproteins, including other Ig classes; agarose-bound Jacalin can be used to distinguish IgA₁ from IgA₂ (Roque-Barreira, MC et al., J. Immunol. 134:1740 (1985); Gregory, R L, J. Immunol. Meth. 99:101 (1987)) because binding is stronger to IgA₁.

Mannan-Binding Lectin, MBL, is a plasma protein (32 kDa molecular mass) structurally related to complement C1, that is secreted by the liver and binds specific mannose-containing carbohydrates on the surface of various microorganisms including bacteria, yeasts, parasitic protozoa, and viruses; activates the complement cascade through MBL-associated serine protease (MASP) and promotes phagocytosis. MBL is an oligomeric complex of 6 set of homotrimers. MBL is a calcium-dependent C-type lectin that binds mannose and GlcNAc in a calcium- dependent manner. Due to the presence of mannose on IgM, this protein binds antibodies of the IgM class.

Lectins as LBPs

Lectins are proteins or glycoproteins, commonly derived from plants or marine animals (lectins from bacteria, viruses, and mammals are also well-known) that have binding specificity for a particular sugar or sugars, usually a mono- or disaccharide structure. For example, Concanvalin A (Con A) binds α-D-Glc and α-D-Man. Lectin binding, like antibody binding to antigen, is noncovalent and reversible (typically by a sufficient concentration of the saccharide ligand. Thus, for example, a solution of glucose or mannose (or α-methylmannoside) will release Con A that has bound to cells or to an immobilized glycoprotein. For thorough description of plant lectins, see, for example, EJM Van Damme et al., Handbook of Plant Lectins: Properties and Biomedical Applications John Wiley & Sons, New York, 1998; see also the web site http ://www.plab.ku.dk/tcbh/ and http://www.vectorlabs.com/Lectins/Lindex.html for commercially available lectins. Other useful reviews include Goldstein, I J et al., 1978, Adv. Carbohydr. Chem. Biochem. 35:127-340; D. Mirelman (ed.), Microbial Lectins and Agglutinins: Properties and Biological Activity, Wiley, N.Y. (1986); Goldstein I J, Indian J Biochem Biophys, 1990,27:368-369.

Lectins can be immobilized directly to the CAR material on the surface, or, as with antibodies, can be used in a sandwich fashion where a first LBP has binding specificity and affinity for the lectin (such as an anti-lectin antibody or streptavidin when the lectin is biotinylated) and the lectin serves as a “second LBP” and is bound noncovalently to the first LBP. The lectin acts as the capture agent to bind its specific target preferably a cell that displays a particular saccharide structure on a cell surface. Typically, such saccharide target structures are in the form of carbohydrate chains on glycoproteins or glycolipids.

Table 2, below lists a number of useful lectins and their sugar-binding specificities.

Also included in the present invention as an LBP is a covalently coupled lectin-antibody or lectin-antigen conjugate (see, e.g., Chu, U.S. Pat. No. 4,493,793).

Yet another class of LBP in the present invention is a basic molecules that has affinity for the lipid bilayer of the cell membrane, for example, protamine and the membrane binding portion of the bee venom peptide, mellitin. While these target structures may not formally be considered “ligands” the concept is the same—affinity capture of cells which bind to this IBP when it is immobilized to a solid surface.

Biotinylated Second LBPs

In one embodiment of this invention, streptavidin as a first LBP is covalently immobilized to the solid phase-bound CAR material, preferably HA. The streptavidin will then bind with high affinity to any biotinylated polypeptide which will serve as the second LBP that will target structures on cells. Examples of biotinylated polypeptides are ECM polypeptide such as collagen, laminin, fibronectin, thrombospondin 1, vitronectin, elastin, tenascin, aggrecan, agrin, bone sialoprotein, cartilage matrix protein, fibrinogen, fibrin, fibulin, mucins, entactin, osteopontin, plasminogen, restrictin, serglycin, SPARC/osteonectin, versican, von Willebrand Factor, and cell adhesion molecules (CAMs), such as cadherins, connexins, and selectins.

In one embodiment, synthetic peptides including the Arg-Gly-Asp (RGD) tripeptide sequence are used as an ECM mimic, since this is the cell attachment domain of many ECM proteins. RGD peptides have been used to modify a number of polymer surfaces (PTFE, polyacrylamide, polyurethanes, and as copolymers with poly(DL-lactic acid co-lysine) (PLA) and poly(DL-lactic-co-glycolic acid) (PLGA), poly(ethylene glycol) acrylic acid copolymers (PEGAA) (discussed in Glass, J R et al., Biomaterials 1 7:1101-1108 (1996) incorporated by reference in its entirety). Glass et al. specifically describe methods for covalently coupling RGD-containing peptides to cross linked HA and created porous 3D matrix of cross-linked HA to which RGD peptides are coupled using periodate oxidation. In the present invention, RGD peptides such as those that are known in the art, are biotinylated while maintaining their bioactivity. These biotinylated peptides (RGD) are allowed to bind to the immobilized streptavidin and constitute an immobilized ECM-like material extending from a CAR surface.

TABLE 1
Immunoglobulin Binding by Proteins A and G,
Jacalin and Mannan Binding Protein

| | | | | | Mannan |
|---|---|---|---|---|---|
| | Protein | | | | Binding |
| Ig Isotype | A | Protein G | Protein A/G | Jacalin | Protein |
| Human IgG | S | S | S | nb | nb |
| Human IgG1 | S | S | S | nb | nb |
| Human IgG2 | S | S | S | nb | nb |
| Human IgG3 | w | S | S | nb | nb |
| Human IgG4 | S | S | S | nb | nb |
| Human IgM | w | nb | w | — | S |
| Human IgA | w | nb | w | S | — |
| Human IgA1 | w | — | S | S | — |
| Human IgA2 | w | — | S | w | — |
| Human IgD | nb | nb | w | — | — |
| Mouse IgG | S | S | S | nb | nb |
| Mouse IgG1 | w | w/s | w/s | — | nb |
| Mouse IgG2a | S | S | S | — | nb |
| Mouse IgG2b | S | S | S | — | nb |
| Mouse IgG3 | S | S | S | — | nb |
| Mouse IgM | nb | nb | nb | — | S |
| Horse IgG | w | S | S | — | — |
| Horse IgG(c) | w | nb | w | — | — |
| Horse IgG(T) | nb | nb | nb | — | — |
| Rabbit IgG | S | S | S | nb | — |
| Goat IgG | w | S | S | — | — |
| Rat IgG | w | w/s | w/s | — | — |
| Sheep IgG | w | S | S | — | — |
| Cow IgG | w | S | S | — | — |
| Guinea Pig IgG | S | w | S | — | — |
| Pig IgG | S | w | S | — | — |
| Dog IgG | S | w | S | — | — |
| Cat IgG | S | w | S | — | — |
| Monkey IgG | S | S | S | — | — |
| (Rhesus) | | | | | |
| Chicken IgG | nb | nb | nb | — | — |

Relative Affinity of Protein G, Protein A, Protein A/G, Mannan Binding Protein and Jacalin for Various Immunoglobulins (as Reported in Literature)
w: Weak binding
S: Strong binding
w/s: Indifferent
nb: No binding
—: Information not available

TABLE 2
Lectins and their Binding Specificity

| Lectin (agglutinin) | Abbrev | Carbohydrate Specificity |
|---|---|---|
| Allium sativum (garlic bulb) | ASA | α(1,3)-linked Man units |
| Arachis hypogaea (peanut) | PNA | Gal(β1,3)-GalNAc |
| Bauhinia purpurea | BPA | GalNAc, Gal |
| Bendeirea simplicifolia | BSA | α-Gal |
| Canavalia ensorformis (jackbean) | Con A | α-Man, α-Glc |
| Crocus vernus (Crocus bulb) | | terminal Man(α1,3)Man |
| Dolichos biflorus (horse gram) | DBA | GalNAc |
| Erythrina cristagalli (coral tree) | ECA | Gal(β1,4)GlcNAc |
| Glycine max (soybean) | SBA | Gal, GalNAc |
| Griffonia simplicifolia-1 | GS-1 | N-linked glycans from murine IgD |
| Griffonia simplicifolia-1-B4 | GS-1-B4 | Gal (α1,3)Gal |
| Griffonia simplicifolia 1-A4 | GS I-A4 | terminal αGalNAc |
| Helix pomatia | HPA | GalNAc |
| Lens culinaris (lentil) | LcH | α-Man, α-Glc |
| Limulus polyhemus (horseshoe | LPA | Sialic Acid (“NeuAc5”) |
| crab) | | |
| Lotus tetragonolobus | Lotus A | α-L-Fucose |
| Marasmius oreades (mushroom) | MOA | Gal(α1,3)Gal |
| Musa acuminata (banana) | BanLec | α-Man; α-Glc (internal α1,3-linked Glc in certain linear |
| | | polysaccharides, β1,3-linked glucosyl oligosaccharides and |
| | | β1,6-linked glucosyl end groups) |
| Phaseolus limensis | LBA I | α-D-GalNAc |
| Phaseolus lunatus (lima bean) | LBL, | GalNAc(α1,3)Fuc(α1,2)Gal(β1,R). |
| Phaseolus vulgaris (red kidney bean) | PHA-L | GalNAc |
| | PHA-H | GalNAc |
| | PHA-E | Oligosaccharide |
| Pisum sativum (pea) | PEA | α-D-Man, α-D-Glc. |
| Phytolacca americana (pokeweed) | PWM | (GlcNAc)₃ |
| Polysporus squamosus (mushroom) | PSL | NeuAc5(α2,6)Gal(β1,4)Glc/GlcNAc (of N-linked oligosacch |
| Ricinus communis (castor bean) | RCA I | β-D-Gal |
| | RCA II | β-D-Gal, D-GalNAc |
| Sambucus nigra (elderberry bark) | SNA | NeuAc5(α2,6)Gal/GalNAc (does not discriminate between |
| | | O-linked and N-linked oligosaccharides |
| Sophora japonica (pagoda tree) | SJA | αGalNAc |
| Triticum vulgaris (wheat germ) | WGA | (GlcNAc)₂; NeuAc5 |
| Ulex Europaeus (Furze gorse) | UEA I | α-L-Fucose |
| | UEA II | (GlcNAc)₂ |
| Wisteria Floribunda (Japanese Wister) | WFA | GalNAc |

These RGD peptides and any other synthetic peptide are biotinylated either after synthesis or during synthesis by use of biotinylated amino acids in the synthetic process.

Other biotinylated polypeptides useful as second LBPs are any of the know growth factors that bind to extracellular receptors (for example, epidermal growth factor, fibroblast growth factors, platelet-derived growth factor, nerve growth factor, transforming growth factor-β, and any of the hematopoietic growth factors or interleukins that stimulate growth of lymphocytes and other immune system cells.

Non-polypeptide molecules that also bind desired targets may be used in place of the second LBP. Here they would be termed “ligand binding molecules” (LBM). Examples of useful second LBMs are glycosaminoglycans which can similarly be biotinylated, nucleic acid molecules (DNA or RNA) including oligonucleotides.

Rather than using avidin-biotin, an antibody specific for any of the above molecules can be immobilized covalently as the first LBP and used to bind these second LBPs (and nonpeptidic LBMs) to the solid surface for use as described herein.

Of course, all of the above peptides, polypeptides, and nonpeptidic molecules can also serve as first LBPs (or LBMs) by their direct bonding to the CAR material. Methods for biotinylation of polypeptides and other macromolecules are well known in the art (Hermanson, G. T., Bioconjugate Techniques. 1996, San Diego: Academic Press). For example, sulfo-NHS biotin may be used. Alternatively, 5-(biotinamido) pentylamine or biotin hydrazide may be the reagent of choice. Those skilled in the art will know which biotinylating agent to select and how to use it for the objectives presented herein.

The amount of bound first or second (or third) LBP bound to the solid surface can be assessed by any know method for measuring a particular polypeptide bound to a polymer or plastic. Any detectably labeled binding partner for the immobilized polypeptide may be added and the amount of binding partner that binds to the surface can be assessed by routine methods appropriate to the label, e.g., by fluorescence, color, or chemiluminescence. This is exemplified below for antibodies using Alexa Fluor 488™, a fluorescently labeled goat anti-mouse Ig.

It should also be noted that the surfaces described herein may be used to culture the cells after they have been captured. Thus, if the form of the surface is appropriate (e.g., dish or flask), cells that have adhered specifically to the LBP may be left in place after nonadherent cells have been removed, and allowed to grow, differentiate, secrete factors, etc. It is expected that only cells which do not require an adhesive surface will grow in such vessels, as the surface has been modified to comprise a CAR substance. Functional or “structural” assays of the cells after such growth may be one way to assess the quality of the original separation or enrichment. Cell growth would likely require the addition of growth factors or ECM molecules that support more physiologic cell attachment when cells detach from the immobilized LBP over time.

In a preferred embodiment, a PS surface of a culture flask is coated with HA (with or without an intermediate layer) and then with an anti-CD34 mAb which is either a first or a second LBP. An unfractionated population of cells which contains (or is suspected of containing) CD34+ cells is added to the surface and allowed to adhere. Such cell populations may be derived from bone marrow, mobilized peripheral blood, placenta or umbilical cord blood. Nonadherent cells are washed off and discarded. The flask is filled to the desired volume with growth medium optimized for growth of hematopoietic progenitor cells. Preferably, ECM materials are added to the medium added after nonadherent cells are removed. The specifically adherent CD34+ are stimulated to grow and may be grown to large numbers for clinical use (e.g., stem cell transplantation). If desired, inducers of specific differentiation pathways may be added to selected cultures to drive differentiation of the progenitor cells along the desired pathway (lymphoid, granulocytoid, monocytoid).

In another embodiment, LBP-coated surfaces described herein are used to bind not intact cells but rather cell lysates or other subcellular preparations.

The CAR surface of the present invention can be prepared with the first or second LBP (the capture agent) distributed in any pattern or array, such as a microarray pattern of dots arranged in preselected patterns on the polymer surface. Thus, for example, microarrays of one or more different types of antibodies may be immobilized to a CAR surface as described herein. In addition to capture or binding or intact cells, the LBP-coated surfaces described herein, for example in the form of a antibody microarray, are used to detect or quantitate any of a number of corresponding antigens or epitopes in a cell lysate or other subcellular preparation. Thus, the present invention provides a method for producing a device comprising a high density array of LBPs, such as antibodies or ligands for cell surface receptors. Such a device may is useful in a method for quantitating expression levels of specific proteins in a cell population, for example, cells treated in vitro in a selected manner to induce differentiation or another cellular activity. These devices and methods can be readily adapted to high throughput analysis of cells treated (or not treated) with a test agent such as a drug. For example, groups of cells treated with various drugs are lysed and the lysates taken, or culture supernatants can be taken, and placed onto CAR surfaces onto which an antibody library microarray or receptor ligand peptide library has been immobilized.

The present method can be used in a “replica plating” or split culture system, where cells are grown in separate wells or attached to distinct regions of a growth surface, treated in some way, observed or tested for a functional response. An aliquot of cells or supernatant from each well, or cells from a particular surface region are then transferred to a corresponding CAR surface of the present invention which displays a microarray of LBP's such as antibodies to test for the present or amount of particular cellular products either expressed on intact cells, secreted from cells or present intracellularly and releasable by some extraction or lysis procedure. This split or replica method permits correlation between, for example, a selected functional activity or activities of a discrete population of cells and its expressed protein products.

In another embodiment in which whole cells are used, the present invention provides a method for interrogating cell surface receptors using a library of immobilized ligands including but not limited to peptides, extracellular matrix molecules, growth factors, cytokines, antibodies, glycosaminoglycans, lectins, and the like. The readout in such a system may be a functional assay, avoiding the use of intracellular reporter genes.

The foregoing methods and devices have many uses as part of an immunodiagnostic and other diagnostic procedure that evaluate either cells or various body fluids.

Basic Immobilization Processes and Options for a Given Ligand Binding Polypeptide

In one general embodiment of the invention, the LBP is immobilized to the CAR substance, preferably HA which is deposited as described herein. Using an anti-CD34 mAb as an example (see also FIGS. 3-7) the process comprises a step of covalently immobilizing anti-CD34 directly onto periodate-activated HA using reductive amination as described herein.

In another embodiment involving a first and a second LBP, protein A or protein G is immobilized to periodate-activated HA. The anti-CD34 mAb is then allowed to bind noncovalently to the Protein A or Protein G.

In another embodiment involving a first and a second LBP, avidin or streptavidin is immobilized to periodate-activated HA. The biotin is conjugated to the anti-CD34 mAb and the biotinylated mAb is allowed to bind to the avidin/streptavidin.

Covalent Coupling of Ligand Binding Polypeptide to an CAR-Coated Surface

Surfaces coated with HA, AA or another such CAR material are described above and in the Examples. Oxidation of these polysaccharides leads to cleavage of the sugar ring between two adjacent hydroxyl groups and the creation of two reactive aldehyde groups. Typically, when a aldehyde moiety (RCHO) reacts with a primary amine moiety (R′NH₂), an equilibrium is established with the reaction product, which is a relatively unstable imine moiety (R′N CHR). This coupling reaction can be carried out under the same conditions described above for the oxidation, which are designed to protect the glycoprotein from damage. To stabilize the linkage between the glycoprotein and the biomaterial surface, subsequent reductive alkylation of the imine moiety is carried out using reducing agents (i.e., stabilizing agents) such as, for example, sodium borohydride, sodium cyanoborohydride, and amine boranes, to form a secondary amine (R′NH—CH₂R). This reaction can also be carried out under the same conditions as for the oxidation. Typically, however, the coupling and stabilizing reactions are carried out in a neutral or slightly basic solution and at a temperature of about 0-50° C. Preferably, the pH is about 6-10, and the temperature is about 4-37° C., for the coupling and stabilizing reactions. These reactions (coupling and stabilizing) can be allowed to proceed for just a few minutes or for many hours. Commonly, the reactions are complete (i.e., coupled and stabilized) within 24 hours.

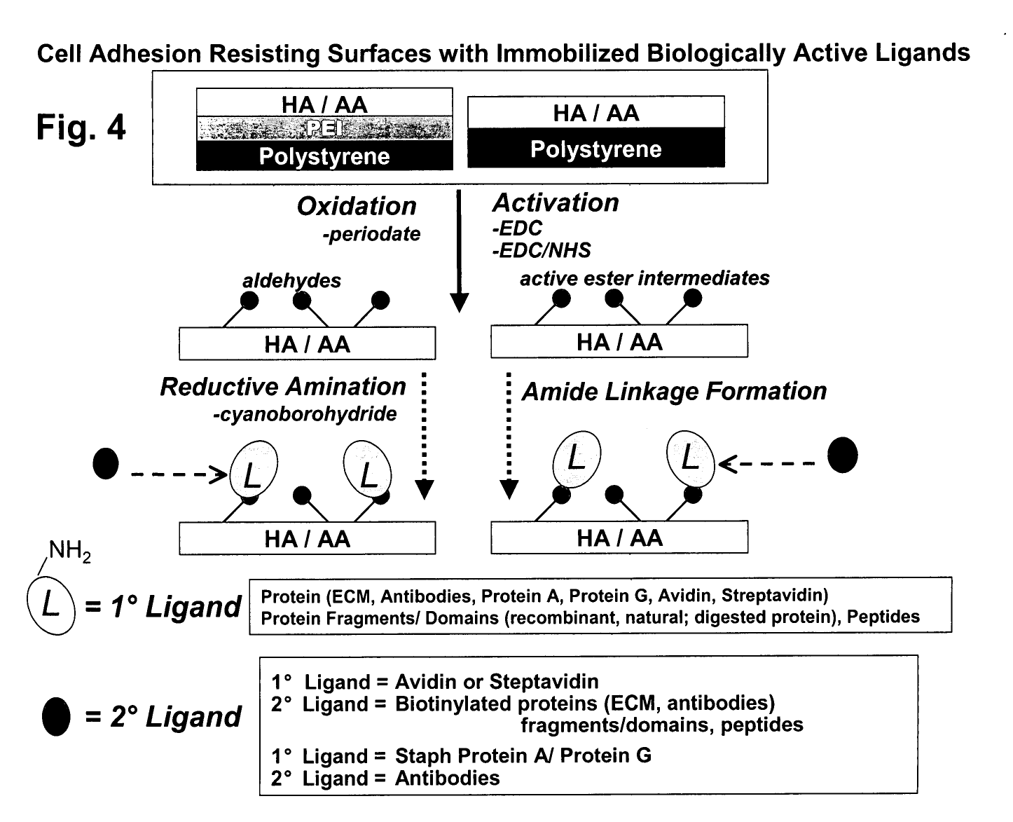

In another method, instead of oxidation, surfaces coated with HA, AA or another such CAR material are treated in a manner similar to that described in the section above on “Coupling Reaction Between Carboxyl Groups on HA and Amine Groups on a Surface.” Here, carboxyl groups on HA (or other CAR material) are reacted with amine groups on the peptide or polypeptides (or other amine-containing molecule) in order to couple the peptide or polypeptide to the HA, and, thus, to the surface. This is described schematically on the right side of FIG. 4. As discussed above the COO⁻ groups of the CAR material, are activated to form reactive intermediate o-acylisourea esters by the addition of EDC. The unstable intermediate is preferably stabilized by the addition to the reaction of NHS, sulfo-NHS or other reactive intermediate ester stabilizing compound. Free amino groups of the peptide or polypeptide react with these intermediate esters to form a stable amide bond, thereby immobilizing the peptide or polypeptide covalently to the surface. These reactions may be carried out in either one or two steps. The two-step reacton involves first treating the HA, AA or other CAR material with EDC with or without the stabilizing compound, and then, as a second step, adding the peptide or polypeptide. In the single step reaction, the components are all combined (surface-bound HA, EDC, optional stabilizing compound and polypeptide), and the bonding allowed to occur.

Having now generally described the invention, the same will be more readily understood through reference to the following examples which are provided by way of illustration, and are not intended to be limiting of the present invention, unless specified.

### EXAMPLE 1

### ESCA Analysis of Untreated and Plasma Treated Polystyrene

60 mm diameter polystyrene petri dishes (Falcon, BD Biosciences) were prepared as follows:
1. Control (untreated)
2. Air plasma treated

The plasma treatment was performed at a pressure of 140 mTorr while bleeding air into the plasma chamber at a constant rate of 8sccm, at 40 W power for a total treatment time of 30 sec.

ESCA (Electron spectroscopy for chemical analysis) was performed to assess the efficacy of the treatment. ESCA was performed on a SSX-

spectrometer (Surface Science Incorporated, Mountain View, Calif.) equipped with a monochromatized Al Kα X-ray source, a hemispherical electron analyzer and a low-energy electron flood gun for charge compensation when studying polymer samples (insulators). Typically, samples were introduced into a preparation chamber which was maintained at about 10⁻⁴ Torr, and then transferred into the analysis chamber, which was typically maintained at 10⁻⁸ Torr. The samples were typically analyzed at an electron take-off angle, defined as the angle between the sample surface and the position of the hemispherical analyzer, of 35° which corresponds roughly to a sampling depth between 50-100 Å.

The resulting surface compositions are shown in Table 3:

TABLE 3

| | C (%) | O (%) | N (%) | S (%) | Cl (%) |
|---|---|---|---|---|---|
| Untreated | 97.07 | 2.93(*) | — | — | — |
| Untreated | 96.89 | 3.11(*) | — | — | — |
| Plasma treated | 82.05 | 17.95 | — | — | — |

(*)Source of oxygen contamination of untreated polystyrene is not known. Theoretically the ESCA spectrum for PS should be 100% carbon.

Oxygen contamination on the untreated dish is unclear but may be caused by small traces of SiO₂ contaminations.

### EXAMPLE 2

### Comparison of Primaria™ and PLL-Treated Surfaces

This Example describes a comparison of as-received Primaria™ surfaces to PLL treated surfaces, unmodified or modified with 0.5% HA using potassium phosphate buffer and EDC only. Stability testing of HA surfaces were performed in ethanol.

60 mm PS petri dishes (Falcon, BD Biosciences) were divided into groups as follows:
1. (2) untreated
2. (2) air plasma treated
3. (2) air plasma treated, PLL modified
4. (2) air plasma treated, PLL modified and HA modified (0.5% HA)
5. (2) air plasma treated, PLL modified and HA modified (0.5% HA), rinsed with ethanol

35 mm Primaria™ dishes were divided into groups as follows:
1. (2) Primaria™
2. (2) Primaria™, and HA modified (0.5% HA)
3. (2) Primaria™, and HA modified (0.5% HA), rinsed with ethanol

Air plasma treatment was performed as described in Example 1 (on non-Primaria™ dishes). Total treatment time was 30 seconds using a steady air inbleed at a rate of 8 sccm resulting in a treatment pressure within the plasma chamber of 140 mTorr.

Poly-L-lysine (PLL) (Sigma) was dissolved in deionized water (DIH₂O) to make a 0.025% solution that was coated onto 5 plasma treated polystyrene dishes by incubating the dishes filled with the polylysine solution in an incubator at 37° C over night. The polylysine solution was removed the following morning and dishes were rinsed thoroughly with DIH₂O and allowed to dry until further modifications.

40 mm diameter Primaria™ dishes were purchased from BD Biosciences (Labware, Bedford) and used as received.

HA coating. A 0.5% HA (from rooster comb, Sigma) solution was prepared in potassium phosphate buffer. EDC was dissolved in potassium phosphate buffer and added to result in a ratio of about 1 EDC molecule per HA repeat unit. PLL-coated and Primaria™ surfaces were coated with 5ml of the HA/EDC solution in potassium phosphate buffer and allowed to stand overnight for reaction to occur. The following morning the HA solution was removed, each dish was washed thoroughly with DIH₂O to remove any non-covalently attached HA and allowed to air dry.

Cell Culture Protocol:

MC3T3-E1 osteoblast cells, originated from Dr. L. D. Quarles, Duke University and kindly provided by Dr. Gayle E. Lester, University of North Carolina at Chapel Hill, were grown in our laboratory using standard cell culture techniques. MC3T3-E1 is a well characterized and rapidly growing osteoblast cell line that was chosen because it attaches aggressively to most commonly used tissue culture surfaces. Other cell lines available to those of skill in the art should produce similar results.

Cells were removed from cell culture flasks using trypsin-EDTA, according to methods known in the art. Cells were enumerated, spun down and resuspended in media containing 10% fetal calf serum. The addition of fetal calf serum at this level makes the test for cell adhesion prevention on the HA coated surfaces more stringent.

Immediately before cell seeding, one HA coated dish from each treatment, e.g., one PLL-coated and Primaria™ surface coated with HA, were soaked for 1 hour in ethanol to investigate the stability of the HA coated surface towards this sterilization method.

Cells were seeded (about 1 million cells per 60 mm dish, 800,000 cells per 35 mm dish) and incubated at 37° C. in an incubator. Cell attachment was monitored by phase contrast microscopy at 30 min, 5h, 20h, 29h, 44h, 5d, 6d and 7d after cell seeding. Cell attachment was scored as indicated below. This scoring system is used throughout the Examples.

Scoring: ++ cells attached and spread, form confluent cell monolayer

+ cells attached and spread, do not form confluent monolayer
± cells attached, not spread (round shape)
- very few cells attached, not spread (round shape)
- - no cells observed
nd notdone

The results are shown in Table 4.

TABLE 4

| Polystyrene Surfaces: | Cell Attachment Score at times: (hrs or days) | | | | | | | |
|---|---|---|---|---|---|---|---|---|
| Treatments and Coatings | 0.5 h | 5 h | 20 h | 29 h | 44 h | 5 d | 6 d | 7 d |
| Untreated | + | ++ | ++ | ++ | ++ | ++ | ++ | ++ |
| Air Plasma | + | ++ | ++ | ++ | ++ | ++ | ++ | ++ |
| Air Plasma, PLL | + | ++ | ++ | ++ | ++ | ++ | ++ | ++ |
| Air Plasma, PLL, | + | + | + | + | + | ++ | ++ | ++ |
| 0.5% HA | | | | | | | | |
| Air Plasma, PLL, | + | + | + | + | + | ++ | ++ | ++ |
| 0.5% HA, | | | | | | | | |
| ethanol rinse | | | | | | | | |
| Primaria ™ surface | + | ++ | ++ | ++ | ++ | ++ | ++ | ++ |
| Primaria ™ surface, | + | ++ | ++ | ++ | ++ | ++ | ++ | ++ |
| 0.5% HA | | | | | | | | |
| Primaria ™ surface, | + | ++ | ++ | ++ | ++ | ++ | ++ | ++ |
| 0.5% HA | | | | | | | | |
| ethanol rinse | | | | | | | | |

In summary, confluent cell layers were formed on PLL and Primaria™ surfaces modified with 0.5% HA in the presence of EDC. The surfaces that were ethanol rinsed demonstrate solvent stability of HA coating—HA coating can be washed with ethanol without changing its performance.

Results of ESCA analysis (see Example 1 for description of ESCA set-up) are shown in Table 5.

TABLE 5

| | C (%) | O (%) | N (%) | S (%) | Cl (%) |
|---|---|---|---|---|---|
| Untreated | 97.24 | 2.76(*) | — | — | — |
| Plasma treated | 85.60 | 14.40 | — | — | — |
| +0.025% PLL | 85.35 | 11.40 | 3.25 | — | — |
| Primaria ™ | 81.00 | 13.51 | 5.49 | — | — |
| +PLL + 0.5% HA | 84.62 | 12.13 | 3.25 | — | — |
| Primaria ™ + 0.5% HA | 79.41 | 14.41 | 5.43 | — | — |

(*)Source of oxygen contamination of untreated polystyrene is not known. Theoretically the ESCA spectrum for PS should be 100% carbon.

Air plasma treatment introduces oxygen-containing groups, whereas nitrogen is introduced by PLL modification and is present at the Primaria™ surface. For poly-L-lysine, one of each two nitrogens represents a functional[primary] amine group suitable for covalently coupling of HA.

The addition of HA is again followed by changes in the O/C and O/N ratios as follows:

TABLE 6

| | O/C | O/N |
|---|---|---|
| Untreated | NA | NA |
| Plasma treated (PT) | 0.17 | NA |
| PT + 0.025% PLL | 0.13 | 3.5 |
| PT + PLL + 0.5% HA | 0.14 | 3.73 |
| Primaria ™ | 0.17 | 2.46 |
| Primaria ™ + 0.5% HA | 0.18 | 2.65 |

The O/C and O/N ratios increased for all surfaces after addition of HA, indicating that some HA was coupled to these surfaces. The lack of cell adhesion prevention observed on PLL or Primaria™, however, indicates that the amount of HA at the surface modified according to the procedure described in this example was not sufficient.

### EXAMPLE 3

### Comparison of Potassium Phosphate and MES Buffer for Coupling HA to PLL treated Surfaces, Using EDC Only

One 96-well flat-bottom microtiter plate was air plasma treated according to the plasma process described in Example 1. Total treatment time was 60 seconds at a steady air inbleed at a rate of 8 sccm resulting in a treatment pressure within the plasma chamber of about 140 mTorr.

PLL (Sigma) was dissolved in DIH20 to make a 0.05% solution that was coated onto the bottom of 9 wells in the air plasma treated 96-well plate for two hours at room temperature. The polylysine solution was then removed and wells were rinsed thoroughly with DIH₂O and the plate was left to dry at room temperature until further modifications.

HA coating was performed as described in Example 2. A 0.5% HA (from rooster comb, Sigma) solution was prepared in 0.1M potassium phosphate buffer, pH 5.3. Similarly, a 0.5% HA solution was prepared in 0.1M MES buffer, pH 3.68. EDC was dissolved in either potassium phosphate buffer or MES buffer and added to either HA in potassium phosphate buffer or HA in MES buffer, respectively, to result in a ratio of about 1 EDC molecule per HA repeat unit. PLL coated surfaces in the 96-well plate were modified by adding 100 μl of HA/EDC solution so that PLL treated surfaces were modified by both HA /EDC in potassium phosphate buffer as well as by HA/EDC in MES buffer (3 repeats per condition). The following morning the HA solution was removed, each well was washed thoroughly with DIH₂O to remove any non-covalently attached HA and plate was allowed to air dry until cell culture.

Cell culture using MC3T3-E1 osteoblast cells was performed as described in Example 2. Cells were seeded at about 10,000 cells per well and incubated at 37° C. in an incubator.

Cell attachment was monitored by phase contrast microscopy at 30 min, 50 min, Id, 2d and 5d after cell seeding. Cell attachment was scored as above. Results appear in Table 7.

TABLE 7

| | Cell Attachment Score | | | | |
|---|---|---|---|---|---|
| Polystyrene Surfaces: | at times: (hrs or days) | | | | |
| Treatments and Coatings | 0.5 h | 0.83 h | 1 d | 2 d | 5 d |
| Air Plasma | ± | ± | ++ | ++ | ++ |
| Air Plasma, PLL Coating | ± | ± | ++ | ++ | ++ |
| Air Plasma, PLL, 0.5% HA/PPB¹ | ± | ± | ++ | ++ | ++ |
| Air Plasma, PLL, 0.5% HA/MES² | ± | ± | + | + | + |

¹PPB = potassium phosphate buffer;
²MES = 2-[N-Morpholino]ethane sulfonic acid . . .

In summary, EDC catalyzed coupling of HA to PLL leads to surfaces that do not prevent cell spreading and growth. However, in combination with MES buffer, cell adhesion is reduced and any observable cell attachment is mainly in the form of clumps and remains that way over 5 days of culture. This observation may be the result of partial HA coating of the surfaces, and the observed cell attachment may be due to defects in the HA coating on the underlying PLL coating (which supports attachment).

### EXAMPLE 4

### Primaria™ HA Coupled Surfaces Catalyzed by EDC and EDC/NHS in MES Buffer

Primaria™ treated 24-well flat bottom plates were purchased from BD Biosciences and used as received.

EDC-supported HA coating was performed as described in Example 2. A 0.5% HA (from rooster comb, Sigma) solution was prepared in 0.1M MES buffer, pH 3.68. EDC was dissolved in MES buffer and added to HA dissolved in MES buffer to result in a ratio of about 1 EDC molecule per HA repeat unit. 10 wells in the Primaria™ plate were modified by adding 3 ml of HA/EDC solution. Plates were allowed to stand overnight for reaction to occur. The following morning the HA solution was removed, each well was washed thoroughly with DIH20 to remove any non-covalently attached HA and plates were allowed to air dry until cell culture.

EDC/NHS-supported HA coating was prepared similarly to HA coating using EDC alone. A 0.5% HA (from rooster comb, Sigma) solution was prepared in 0.1M MES buffer, pH 3.68. EDC and NHS were dissolved in MES buffer and added to HA dissolved in MES buffer to result in a ratio of about 1 EDC molecule and 0.5 NHS molecules per HA repeat unit. 10 wells in the Primaria™ plate were modified by adding 3 ml of HA/EDC/NHS solution. Plates and dishes were allowed to stand overnight for the reaction to occur. The following morning the HA solution was removed, each dish and well were washed thoroughly with DIH₂O to remove any non-covalently attached HA and plates and dishes were allowed to air dry until cell culture.

Cell culture using MC3T3-E1 osteoblast cells was performed as described in Example 2.

Cells were seeded at different seeding densities, e.g. 100, 250, 1000, and 2000 cells per mm² of culture surfaces onto Primaria™ treated, Primaria™ treated, HA/EDC modified, and Primaria™ treated, HA/EDC/NHS modified surfaces in the 24-well Primaria™ plate. 2.5 hours after cell seeding, media and any non-adherent cells were removed from Primaria™ treated, Primaria™ treated and HAIEDC modified, and Primaria™ treated and HA/EDC/NHS modified surfaces. Surfaces with any adherent cells were washed gently once with media and 2 ml media were placed on surface to maintain any adherent cells for the duration of the study.

All cultures were maintained in a 37° C. incubator between microscopy studies. Cell attachment was monitored by phase contrast microscopy at 30 min, 2h, Id, 2d and 5d after cell seeding. Cell attachment was scored as above. The results are shown in Table 8.

These exemplary results clearly demonstrate that cell attachment was prevented on HA surfaces prepared using a medium containing MES buffer with a combination of EDC and NHS. Adding NHS at a ratio of about 0.5 NHS molecules per HA repeat unit is sufficient to stabilize the reactive intermediate and increase the yield of the HA coupling reaction even in the absence of an intermediate layer such as PLL or PEI which has been previously believed necessary, if the surface has been plasma-treated so that sufficient amine groups are present.

TABLE 8

| | Cell Attachment Score | | | | |
|---|---|---|---|---|---|
| Cell Density on surface | at times: (hrs or days) | | | | |
| (cells/mm²) | 0.5 h | 2 h | 1 d | 2 d | 5 d |
| Untreated Primaria ™ | | | | | |
| 100 | ± | + | + | + | + |
| 100, washed¹ | ± | + | + | + | + |
| 250 | ± | + | + | + | + |
| 250, washed | ± | + | + | + | + |
| 500 | + | + | ++ | ++ | ++ |
| 500, washed | + | + | ++ | ++ | ++ |
| 1000 | + | ++ | ++ | ++ | ++ |
| 1000, washed | + | ++ | ++ | ++ | ++ |
| 2000 | + | ++ | ++ | ++ | ++ |
| 2000 washed | + | ++ | ++ | ++ | ++ |
| Primaria ™ 0.5% HA/EDC | | | | | |
| 100, washed | − | − | − | + | + |
| 250, washed | − | − | − | + | + |
| 500, washed | − | − | − | + | + |
| 1000, washed | − | − | − | + | ++ |
| 2000, washed | − | ± | ± | + | ++ |
| Primaria ™ | | | | | |
| 0.5% HA/EDC/NHS | | | | | |
| 100, washed | − | − − | − − | − − | − − |
| 250, washed | − | − − | − − | − − | − − |
| 500, washed | − | − − | − − | − − | − − |
| 1000, washed | − | − − | − − | − − | − − |
| 2000, washed | − | − − | − − | − − | − − |

¹“washed” means that non-adherent cells were removed after 2.5 hours, the surface rinsed with PBS, and replenished with medium.

### EXAMPLE 5

### Ammonium Plasma Treatment Followed by Hyaluronic Acid Coating after 6 Days

Additional experiments were carried out with the goal to identify plasma treatment conditions, in addition to Primaria™, that allow direct covalent attachment of a cell-adhesion resisting layer of either hyaluronic acid (HA) or alginate (AA) to polymer surfaces.

60 mm polystyrene dishes (Falcon 1007, BD Labware) were subjected to five different ammonia plasma treatments (four dishes per treatment condition). All treatments employed a 95 watt, 13.56 MHz RF planar diode generated plasma with the samples placed on the bottom driven electrode with the top electrode grounded. The electrodes were 20 cm in diameter and 6 cm apart. The treatment conditions were:
Condition A: Chamber pumped to a 20 mTorr base pressure, a 375 mTorr NH₃ atmosphere established, and a 25 sec plasma treatment given.
Condition B: Chamber pumped to a 20 mTorr base pressure, a 375 mTorr NH₃ atmosphere established, and a 120 sec plasma treatment given.
Condition C: Chamber pumped to a 0.3 mTorr base pressure, a 200 mTorr argon atmosphere established, a 60 sec plasma treatment given, followed by a 25 sec, 375 mTorr NH₃ plasma treatment.
Condition D: Chamber pumped to a 0.3 mTorr base pressure, a 200 mTorr argon atmosphere established, a 60 sec plasma treatment given, followed by a 120 sec, 375 mTorr NH₃ plasma treatment.
Condition E: Chamber pumped to a 20 mTorr base pressure, a 360 mTorr atmosphere established comprised of 17% argon and 83% NH₃, and a 25 sec plasma treatment given.

Following plasma treatment, samples were stored at room temperature (RT) for 6 days before coating them with HA according to the following procedure.

ESCA Analysis

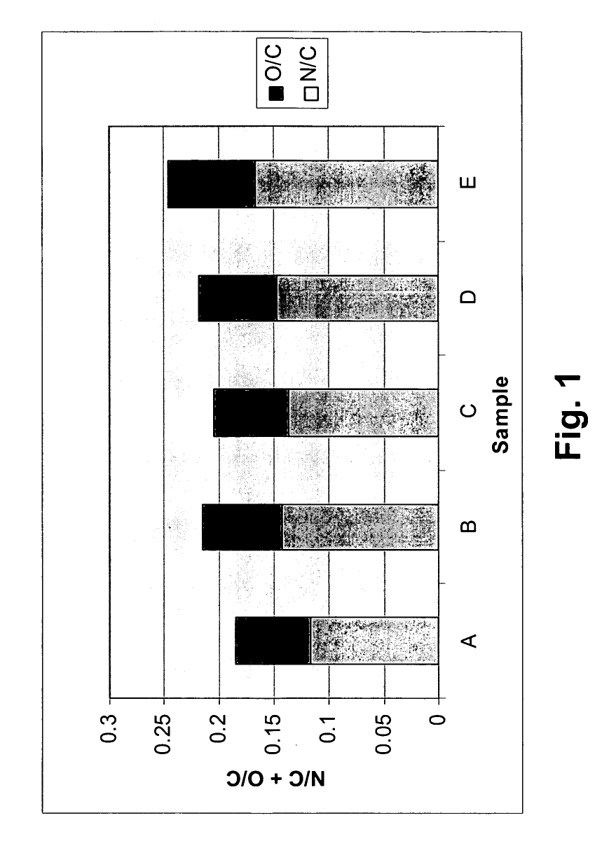

Electron Spectroscopy for Chemical Analysis (ESCA) was used to study the chemical composition of the ammonia-plasma treated polystyrene dishes. All plasma treated dishes showed carbon, oxygen, nitrogen, and some showed small amounts (<1%) of Cl contamination. N/C and O/C ratios shown in FIG. 1.

HA-Coating Procedure:

Plasma treated dishes were HA coated according to the procedure described in Example 4.

Cell Culture:

MC3T3-E1 osteoblast cells, treated as described above, were seeded into dishes at a seeding density of about 700 cells per mm² culture surface. After about 4.5 hours of incubation, phase contrast images of the live cultures were obtained. Cell attachment was seen on all surfaces.

However, cells on plasma-treated controls had formed a confluent monolayer by that time while cell attachment to plasma treated and HA-coated dishes was still patchy with areas showing no cell attachment on the dish surface.

Cell attachment to the plasma treated and HA-coated surface was scored as described above, and the results are presented in Table 9.

TABLE 9
Cell Attachment to PS Surfaces Coated with HA either
15 minutes or 6 days after Ammonia-Plasma-treatment

CELL ATTACHMENT SCORE

| | | HA coating at | HA coating at |
|---|---|---|---|
| Sample ID | Coating | 15 min | 6 days |
| Sample A | — | ++ | ++ |
| | HA | −− | + |
| Sample B | — | ++ | ++ |
| | HA | −− | + |
| Sample C | — | nd | ++ |
| | HA | nd | + |
| Sample D | — | nd | ++ |
| | HA | nd | + |
| Sample E | — | ++ | ++ |
| | HA | − | + |

Cell adhesion was not prevented but was significantly reduced by coating HA using the disclosed procedure on ammonia-plasma treated dishes when the dishes had been “aged” for 6 days between plasma treatment and HA coating.

### EXAMPLE 6

### Ammonium Plasma Treatment Followed by Hyaluronic Acid Coating after 15 Minutes

60 mm polystyrene dishes (Falcon 1007, BD Labware) were subjected to plasma treatment Condition A, Condition B and Condition E (four dishes per treatment condition) selected from the five plasma treatment conditions described in Example 5.

ESCA Analysis:

ESCA analysis was performed on the samples, as described above. All plasma treated dishes showed carbon, oxygen, nitrogen, and some showed small amounts (<1%) of Cl contamination, as in Example 5.

HA-Coating Procedure:

Following plasma treatment, samples were immediately coated (within 15 minutes) with HA according to the procedure set forth in Example 5.

Cell Culture:

MC3T3-E1 osteoblast cells were removed from cell culture flasks using trypsin-EDTA. Cells were enumerated, spun down and resuspended in media containing 10% fetal calf serum. Cells were seeded into dishes at a seeding density of about 420 cells/mm². After about 3 hours of incubation, phase contrast images of the live cultures were obtained and cell attachment to the plasma treated and HA coated surface was scored again according as described in the legend to the Table which summarizes the results. . Cell attachment was seen on all plasma treated control surfaces. However, rounded cells which appeared not to be adhering were found on all plasma-treated, HA-coated dishes.

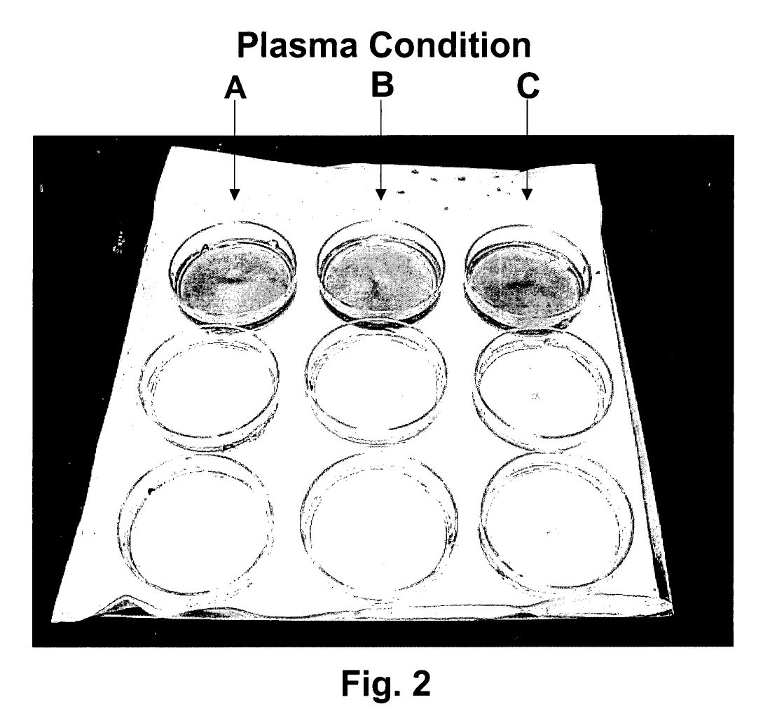

Following phase contrast microscopy, the media and any non-adherent cells were removed, dishes rinsed 2 times with PBS and cells were fixed by incubation with 3 ml of a 10% formalin solution (Sigma) overnight. Formalin was removed the next morning, dishes were rinsed twice in PBS and cells were stained with 2 ml hematoxylin solution per dish. FIG. 2 compares staining in all dishes. Purple stained cells were visible in all three plasma control dishes. In contrast, no purple staining was observed in dishes that had been plasma-treated and HA coated using Condition A and B. Little staining was visible in dishes plasma treated using Condition E followed by HA coating, indicating that this coating procedure resulted in a small defects in the HA coating that allowed cells to attach to the underlying polystyrene substrate. These results confirm the observation on the live cultures that cell adhesion was prevented by coating HA using the disclosed procedure on ammonia-plasma treated dishes immediately following plasma treatment. This unexpected finding allows the formation of superior cell adhesion resistant surfaces with hyaluronic acid without the necessity for an intermediate layer of PEI, polylysine, or other amine containing compound, thereby greatly simplifying the production of labware and other articles and devices on which such surfaces are needed. For optimal results, HA treatment of such surfaces should be performed immediately after plasma treatment; however, it has been found that delays of up to about one hour will produce acceptable results.

HA coated surfaces in accordance with the invention are preferably stored dry at 4° C. and are known to retain their cell adhesion resistance for at least five months when stored at these conditions.

### EXAMPLE 7

### Cell Adhesion Resistant Surface to which Anti-CD34 is Immobilized

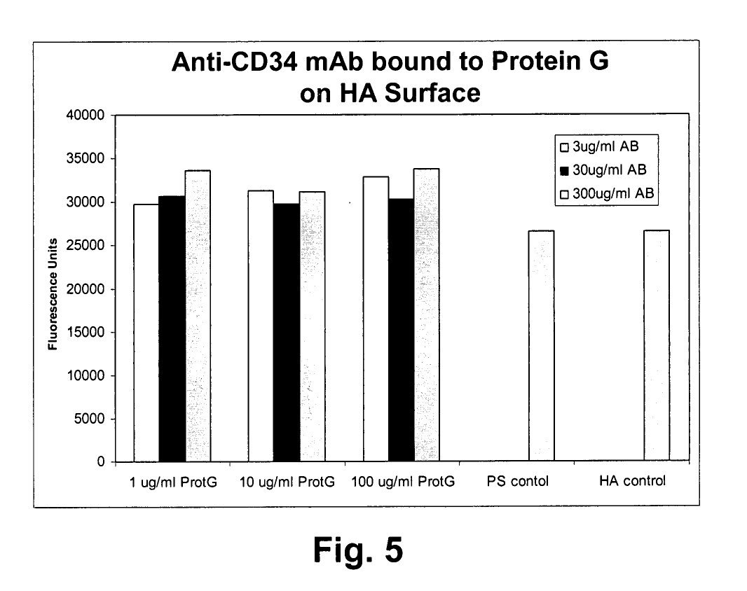

Polystyrene 96 well microplates having HA bound to their surfaces were treated as described below. Adjacent hydroxyl groups in the glucoronic acid ring of the HA were oxidized by treating the covalently coupled HA layer with a 50 mM sodium periodate solution (100 μl/ well for 2 hours). The oxidation reaction leads to cleavage of the glucoronic acid ring and formation of reactive aldehydes. A first LBP in the form of either Protein A, Protein G or avidin (ImmunoPure Avidin (Pierce)) was added to the wells at the concentrations indicated in FIGS. 5-7 and in the presence of cyanoborohydride buffer (Sigma), bonded covalently to the HA. Finally a murine anti-CD34 mAb (IgG1, κ) from Pharmingen was added to the immobilized Protein A or Protein G, and a biotin-conjugated rat anti-mouse CD34 monoclonal antibody (PharMingen) was added to the immobilized avidin at the concentrations shown in FIGS. 5-7. The antibodies were allowed to react with the LBP (SpA, SpG or avidin) overnight at 4° C.

The amount of antibody bound was tested by adding to each well a fixed amount of 10 μg/ml of fluorescently labeled goat anti-mouse IgG (Alexa Fluor 488 goat anti-mouse IgG (H+L) from Molecular Probes) for 1 hour at 4° C. After washing, the fluorescence in the wells was read using a BMG microfluorimeter. The results presented in FIG. 5-7 are direct fluorescence measurements.. These values reflect the amount of antibody bound to the HA-coated surface. Three surfaces were evaluated for binding of anti-CD34 mAb.

Surface 1 (FIG. 5) was HA modified with Protein G (SpG) at three concentrations, e.g., 1, 10, and 100 μg/ml. Anti-CD34 mAb was added to each of these surfaces at 3, 30, and 300 μg/ml, resulting in nine different SpG/Anti-CD34 mAB combinations Miscellaneous controls were included in this experiment, as described with Table below.

Surface 1 included the following groups

| C1 | PS Control |
|---|---|
| 1 | 1 μg/ml SpG + 3 μg/ml anti-CD34 mAb |
| 2 | 1 μg/ml SpG + 30 μg/ml anti-CD34 mAb |
| 3 | 1 μg/ml SpG + 300 μg/ml anti-CD34 mAb |
| 4 | 10 μg/ml SpG + 3 μg/ml anti-CD34 mAb |
| 5 | 10 μg/ml SpG + 30 μg/ml anti-CD34 mAb |
| 6 | 10 μg/ml SpG + 300 μg/ml anti-CD34 mAb |
| 7 | 100 μg/ml SpG + 3 μg/ml anti-CD34 mAb |
| 8 | 100 μg/ml SpG + 30 μg/ml anti-CD34 mAb |
| 9 | 100 μg/ml SpG + 300 μg/ml anti-CD34 mAb |
| C2 | HA, periodate activated, fluorescent 2° Ab |
| C3 | HA, periodate activated, 300 μg/ml anti-CD34 mAb, |
| | fluorescent 2° Ab |
| C4 | HA, periodate activated, 100 μg/ml SpG, fluorescent 2° Ab |
| C5 | HA, periodate activated, 100 μg/ml SpG |
| C6 | HA, fluorescent 2° Ab |
| C7 | HA, 300 μg/ml anti-CD34 mAb, fluorescent 2° Ab |
| C8 | HA, 100 μg/ml SpG, fluorescent 2° Ab |
| C9 | HA, 100 μg/ml SpG |
| C10 | HA only control |

The microplate plate layout is shown below.

| | 1 | 2 | 3 | 4 | 5 | 6 | 7 | 8 | 9 | 10 | 11 | 12 |
|---|---|---|---|---|---|---|---|---|---|---|---|---|
| A | C1 | C1 | C1 | C1 | C1 | C1 | C1 | C1 | C1 | C1 | C1 | C1 |
| B | | 1 | 1 | 1 | 4 | 4 | 4 | 7 | 7 | 7 | C10 | |
| C | | 2 | 2 | 2 | 5 | 5 | 5 | 8 | 8 | 8 | C10 | |
| D | | 3 | 3 | 3 | 6 | 6 | 6 | 9 | 9 | 9 | C10 | |
| E | | C2 | C3 | C4 | C5 | C6 | C7 | C8 | C9 | C10 | C10 | |
| F | | C2 | C3 | C4 | C5 | C6 | C7 | C8 | C9 | C10 | C10 | |
| G | | | | | | | | | | | | |
| H | | | | | | | | | | | | |

The fluorescence results obtained for anti-CD34 mAb immobilized using SpG as the LBP indicated that anti-CD34 mAb was immobilized, but relatively independently of SpG and of the mAb concentration (in the range studied here).

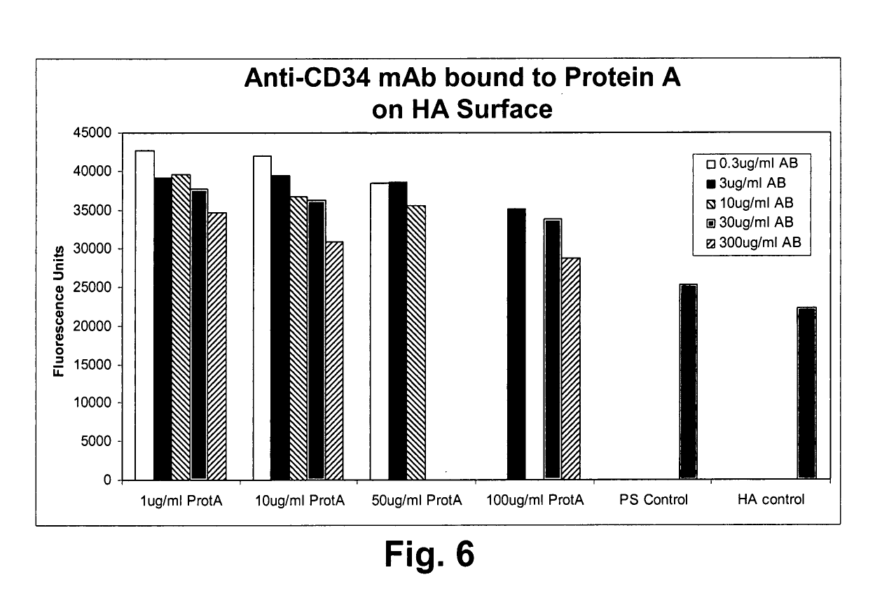

Surface 2 (FIG. 6) was HA modified with SpA at four concentrations: 1, 10, 50, and 100 μg/ml. Anti-CD34 mAb was added to these surfaces at either 0.3, 3, 10, 30, or 300 μg/ml. These experiments were performed in two different 96-well plates and each data point was based on 3 replicates. The different conditions and miscellaneous controls are summarized in the Table below. Surface 2 included the following groups:

| | |
|---|---|
| 1 | 1 μg/ml SpA + 0.3 μg/ml anti-CD34 mAb |
| 2 | 1 μg/ml SpA + 3 μg/ml anti-CD34 mAb |
| 3 | 1 μg/ml SpA + 10 μg/ml anti-CD34 mAb |
| | 1 μg/ml SpA + 30 μg/ml anti-CD34 mAb |
| | 1 μg/ml SpA + 300 μg/ml anti-CD34 mAb |
| 4 | 10 μg/ml SpA + 0.3 μg/ml anti-CD34 mAb |
| 5 | 10 μg/ml SpA + 3 μg/ml anti-CD34 mAb |
| 6 | 10 μg/ml SpA + 10 μg/ml anti-CD34 mAb |
| | 10 μg/ml SpA + 30 μg/ml anti-CD34 mAb |
| | 10 μg/ml SpA + 300 μg/ml anti-CD34 mAb |
| 7 | 50 μg/ml SpA + 0.3 μg/ml anti-CD34 mAb |
| 8 | 50 μg/ml SpA + 3 μg/ml anti-CD34 mAb |
| 9 | 50 μg/ml SpA + 10 μg/ml anti-CD34 mAb |
| | 100 μg/ml SpA + 3 μg/ml anti-CD34 mAb |
| | 100 μg/ml SpA + 30 μg/ml anti-CD34 mAb |
| | 100 μg/ml SpA + 300 μg/ml anti-CD34 mAb |
| C2 | HA, periodate activated, fluorescent 2° Ab |
| C3 | HA, periodate activated, 10 μg/ml anti-CD34 mAb, |
| | fluorescent 2° Ab |
| C4 | HA, periodate activated, 50 μg/ml SpA, fluorescent 2° Ab |
| C5 | HA, periodate activated, 50 μg/ml SpA |
| C6 | HA, fluorescent 2° Ab |
| C7 | HA, 10 μg/ml anti-CD34 mAb, fluorescent 2° Ab |
| C8 | HA, 50 μg/ml SpA, fluorescent 2° Ab |
| C9 | HA, 50 μg/ml SpA |
| C10 | HA only control |

The results obtained with anti-CD34 mAb and immobilized SpA as the LBP indicated that the anti-CD34 mAb was immobilized, and the immobilization appeared to be more efficient at lower SpA and anti-CD34 mAb concentration (over the range studied).

Surface 3 was HA modified with avidin at 3 concentrations: 1, 10 and 100 μg/ml. Again, the presence of the immobilized mAb was detected using a fluorescent anti-immunoglobulin antibody as described above. Surface 3 included the following groups

| | |
|---|---|
| 1 | 1 μg/ml Avidin + 3 μg/ml anti-CD34 mAb |
| 2 | 1 μg/ml Avidin + 30 μg/ml anti-CD34 mAb |
| 3 | 1 μg/ml Avidin + 300 μg/ml anti-CD34 mAb |
| 4 | 10 μg/ml Avidin + 3 μg/ml anti-CD34 mAb |
| 5 | 10 μg/ml Avidin + 30 μg/ml anti-CD34 mAb |
| 6 | 10 μg/ml Avidin + 300 μg/ml anti-CD34 mAb |
| 7 | 100 μg/ml Avidin + 3 μg/ml anti-CD34 mAb |
| 8 | 100 μg/ml Avidin + 30 μg/ml anti-CD34 mAb |
| 9 | 100 μg/ml Avidin + 300 μg/ml anti-CD34 mAb |
| C2 | HA, periodate activated, fluorescently labeled Ab |
| C3 | HA, periodate activated, 300 μg/ml anti-CD34 mAb, |
| | fluorescent 2° Ab |
| C4 | HA, periodate activated, 100 μg/ml Avidin, fluorescent 2° Ab |
| C5 | HA, periodate activated, 100 μg/ml Avidin |
| C6 | HA, fluorescent 2° Ab |
| C7 | HA, 300 μg/ml anti-CD34 mAb, fluorescently labeled Ab |
| C8 | HA, 100 μg/ml Avidin, fluorescently labeled Ab |
| C9 | HA, 100 μg/ml Avidin |
| C10 | HA only control |

The results which reflect binding of the biotinylated anti-CD34 mAb to the immobilized avidin as the LBP indicated that anti-CD34 mAb was immobilized very effectively and the immobilization occurred more efficiently at higher avidin and biotinylated mAb concentrations over the concentration range examined.

To summarize the above observations, SpG was found to be the least efficient LBP of the three tested for immobilizing an antibody to an HA coated surface. In fact, the direct immobilization of the mAb on HA (control wells in these experiments) was greater than the antibody binding to SpG that was fist immobilized on the HA (results not shown).

Binding (immobilization) of the anti-CD34 mAb to SpA covalently bonded to HA was more effective than binding to SpG. Using lower amounts of SpA and anti-CD34 mAb in solution seemed to favor higher immobilization efficiency.

Covalently coupling of avidin to HA and immobilization of biotinylated mAb to this covalently bonded avidin resulted in the largest amount mAb bound to the surface. Higher concentrations of avidin combined with higher concentrations of the biotinylated anti-CD34 mAb resulted in increased fluorescence, indicating more efficient mAb immobilization.

The results shown here indicate that the avidin-biotinylated mAb immobilization strategy resulted in superior antibody binding to an HA coated surface.

### EXAMPLE 8

### Coupling of LBPs to an HA Surface Using EDC/NHS Coupling

Polystyrene 96 well microplates having HA bound to their surfaces were treated using three different chemistries to couple different collagens to the HA as described below.

Periodate Oxidation

Adjacent hydroxyl groups in the glucuronic acid ring of the HA were oxidized by treating the covalently coupled HA layer with 50 mM sodium periodate (50 μl/well) for 2 hours. This oxidation reaction leads to cleavage of the glucuronic acid ring between adjacent ring OH groups and formation of reactive aldehydes.

After oxidation, 50 μl of a solution containing either Collagen Type I, Type III, or Type IV, dissolved at a 100 μg/ml in 10 mM acetic acid buffer, were added to the appropriate wells to react with periodate-oxidized HA surfaces. 50 μl cyanoborohydride buffer (Sigma) were added over the collagen solutions to each well. The plates were incubated overnight. Thereafter, 50 μl Tris (GIBCO) was added to each well and incubated for 2 hrs to block any non-reacted aldehyde groups. After that, solutions were removed from the wells, and the coated surfaces were first washed with a mixture of NaCi, acetic acid and deionized, distilled water (DIH₂0) to remove any ionically bound collagen, followed by at least 3 washes with DIH₂O. Surfaces were left to dry and stored at 4° C. until use in the experiment described below.

EDC/NHS Pre-Treatment

Carboxylate groups on the immobilized bound HA was activated by adding to each well 30 μl of a solution containing EDC and NHS, both at a concentration of 5 mg/ml, in MES buffer (pH 3.6), for 20 minutes. The EDC/NHS solution was then removed, and 100 μl of a 50 μg/ml solution of either Collagen Type I, III, or IV were added to wells and left to react over night. Thereafter, solutions were removed, the wells washed with the NaCl/aceticacid/DIH₂O mixture, followed by at least 3 washes with DIH₂O. Blocking was not necessary in this case because the hydrolytic degradation of the NHS-stabilized EDC produced reactive intermediate ester with time. Coated surfaces were dried and stored at 4° C. until use in experiments described below.

Simultaneous EDC/NHS Activation and Protein Coupling (“Co-treatment”)

A solution containing EDC and NHS, each at a concentration of 5 mg/ml, in MES buffer (pH 3.6), was added to solutions of Collagen Type I, III, or IV to obtain EDC/NHS containing protein solutions with a final protein concentrations of 50 μg/ml. 100 μl of this solution was added per well and left to react overnight. Thereafter, solutions were removed, wells washed with the NaCl/aceticacid/DIH2O mixture, followed by at least 3 washes with DIH₂O. Blocking was not necessary in this case because the hydrolytic degradation of the NHS-stabilized EDC produced reactive intermediate ester with time. Coated surfaces were dried and stored at 4° C. until use in experiments described below.

Results

MC3T3-E1 osteoblast cells, treated as described above, were seeded into wells at a density of about 104 cells/well. Cells were allowed to attach and spread over night. Cells were then stained using Calcein, which stains the cytoplasm of viable cells, and a count of viable cells in each well was obtained using automated microscopy. The numbers of cells adhering to different surfaces (3 different collagens coupled to the HA that was bonded to the PS surface) are shown in Table 10 below.

In control wells having a CAR surface (HA was bonded to PS with no proteins added) no cell attachment and spreading was observed The results of the protein coated HA surfaces are shown below.

TABLE 10
Number of Cells (per well) Adhering to Treated HA Surface

| Protein coupled to | Periodate | EDC/NHS | EDC/NHS |
|---|---|---|---|
| HA coated PS | Oxidation | Pretreatment | Co treatment |
| Collagen Type I | 473 | 280 | 197 |
| Collagen Type III | 375 | 190 | 368 |
| Collagen Type IV | 456 | 317 | 152 |

Viable adherent cells were observed on all protein-coupled surfaces, indicating that both chemistries, i.e., periodate oxidation and carboxylate activation successfully coupled proteins to HA hyaluronic acid (and would be similarly expected to bind proteins to other CAR materials including AA, and to HA and AA derivatives. Proteins covalently coupled to a CAR surface, produced in this manner, permit cells to maintain biological function, in this case, adhesion to a surface.

All the references cited above are incorporated herein by reference in their entirety, whether specifically incorporated or not.

The embodiments illustrated and discussed in the present specification are intended only to teach those skilled in the art the best way known to the inventors to make and use the invention, and should not be considered as limiting the scope of the present invention. The exemplified embodiments of the invention may be modified or varied, and elements added or omitted, without departing from the invention, as appreciated by those skilled in the art in light of the above teachings. It is therefore to be understood that, within the scope of the claims and their equivalents, the invention may be practiced otherwise than as specifically described.

1-58. (canceled)

59. A process of coating a surface of an article with hyaluronic acid (HA) or alginic acid (AA) or a derivative of HA or AA, which consists essentially of the steps of
i) treating a surface of an article with a plasma that causes formation of nitrogen containing groups on said surface; and
ii) exposing the treated surface of the article to a solution containing hyaluronic acid or alginic acid, or a derivative thereof, in the presence of a carbodiimide and a reactive intermediate ester stabilizing compound;
to obtain a coated surface.

60. The process of claim 59 wherein the reactive intermediate ester stabilizing compound is selected from the group consisting of N-hydroxysuccinimide, hydroxysulfosuccinimide and hydroxybenzotriazolohydrate.

61. The process of claim 59 comprising the further step of allowing the coated surface to dry.

62. The process of claim 59 wherein the surface is selected from the group consisting of polystyrene, polyethylene, polypropylene, polyethylene terephthalate, polytetrafluoroethylene, polylactide and cellulose.

63. The process of claim 62 wherein the surface is polystyrene.

64. The process of claim 59 wherein step (ii) is carried out within 60 minutes of step (i).

65. The process of claim 59 wherein the plasma treatment is an NH₃ plasma treatment.

66. The process of claim 65 wherein the plasma treatment is carried out by placing the polymer article into the plasma chamber, evacuating the chamber to a 20 mTorr base pressure, establishing a 375 mTorr NH₃ atmosphere, followed by a 25 sec plasma treatment.

67. The process of claim 59 wherein the concentration of HA is at least about 0.05% w/v.

68. The process of claim 67 wherein the concentration of HA is at least about 0.5% w/v.

69. The process of claim 59 wherein the ratio of carbodiimide and ester stabilizing compound-to-HA repeat unit is at least 1 and 0.5.

70. A process of coating a plasma-treated surface of an article, which surface comprises nitrogen-containing groups, with HA or AA or a derivative of HA or AA, said process comprising:
exposing the plasma-treated surface to a solution of HA, AA or said derivative in the presence of a carbodiimide and a reactive intermediate stabilizing compound, to obtain a coated surface.

71. The process of claim 70 wherein the reactive intermediate ester stabilizing compound is selected from the group consisting of N-hydroxysuccinimide, hydroxysulfosuccinimide and hydroxybenzotriazolohydrate.

72. The process of claim 70 comprising the further step of allowing the coated surface to dry.

73. The process of claim 70 wherein the surface is selected from the group consisting of polystyrene, polyethylene, polypropylene, polyethylene terephthalate, polytetrafluoroethylene, polylactide and cellulose.

74. The process of claim 73 wherein the surface is polystyrene.

75. The process of claim 70 wherein the plasma treated surface is an NH₃ plasma treated surface.

76. The process of claim 70 wherein the article is Primaria™ treated.

77. The process of claim 70 wherein the plasma treatment is carried out by placing the polymer article into the plasma chamber, evacuating the chamber to a 20 mTorr base pressure, establishing a 375 mTorr NH₃ atmosphere, followed by a 25 sec plasma treatment.

78. The process of claim 70 wherein the concentration of HA is at least about 0.05% w/v.

79. The process of claim 78 wherein the concentration of HA is at least about 0.5% w/v.

80. A method for producing a cell-adhesion resistive (CAR) solid phase surface to which is covalently bonded at least a first ligand binding polypeptide, comprising the steps of:
(a) coating a polymer surface with HA, AA, or derivative in accordance with claim 59;
(b) oxidizing said HA, AA or derivative to create an amine-reactive group; and
(c) exposing said oxidized HA, AA or derivative to a first ligand-binding polypeptide wherein covalent bonds are formed between amino groups of said polypeptide and said amine-reactive group, resulting in the covalent bonding of said polypeptide to the HA, AA or derivative,
thereby producing said CAR surface to which is covalently bonded first ligand-binding polypeptide.

81. The method of claim 80, wherein
(i) said oxidizing step (b) is performed by providing an oxidizing agent that generates reactive aldehyde groups on said HA, AA or derivative, and
(ii) step (c) additionally comprises providing a reducing agent to said polypeptide and said surface that effects reductive amination that results in said covalent bond formation between said amino groups of said polypeptide and said reactive aldehyde groups.

82. The method of claim 80, wherein, step (b) additionally comprises, either before step (c) or contemporaneously therewith, the step of converting carboxylate groups of said HA, AA or derivative to reactive esters by exposure to a carbodiimide and a reactive intermediate ester stabilizing compound

83. The method of claim 82 wherein the reactive intermediate ester stabilizing compound is selected from the group consisting of N-hydroxysuccinimide, hydroxysulfosuccinimide and hydroxybenzotriazolohydrate.

84. The method of claim 80, further comprising, after step (c),
(d) contacting said covalently bonded first ligand binding polypeptide with a second ligand binding polypeptide that is a ligand for said first ligand binding polypeptide under conditions that result in the noncovalent binding of said second polypeptide to said first polypeptide.

85. The method of claim 81, further comprising, after step (c),
(d) contacting said covalently bonded first ligand binding polypeptide with a second ligand binding polypeptide that is a ligand for said first ligand binding polypeptide under conditions that result in the noncovalent binding of said second polypeptide to said first polypeptide.

86. The method of claim 82, further comprising, after step (c),
(d) contacting said covalently bonded first ligand binding polypeptide with a second ligand binding polypeptide that is a ligand for said first ligand binding polypeptide under conditions that result in the noncovalent binding of said second polypeptide to said first polypeptide.

87. The method of claim 80, wherein said surface is selected from the group consisting of polystyrene, polyethylene, polypropylene, polyethylene terephthalate, polytetrafluoroethylene, polylactide and cellulose.

88. The method of claim 81, wherein said oxidizing agent of step (b) is periodate.

89. The method of claim 80, wherein said first ligand-binding polypeptide is (a) an antibody, (b) a receptor, (c) an immunoglobulin binding protein, (d) avidin or streptavidin, (e) a lectin, (f) a cell adhesion molecule or (f) an extracellular matrix protein or (g) a synthetic peptide.

90. The method of claim 89 wherein said first ligand-binding polypeptide is an immunoglobulin-binding protein selected from the group consisting of a native or recombinant staphylococcal protein A, a native or recombinant staphylococcal protein G, and recombinant protein A/G.

91. The method of claim 89 wherein said first ligand-binding polypeptide is (a) an antibody or antigen-binding fragment thereof, (b) an immunoglobulin binding protein, or (c) avidin or streptavidin.

92. The method of claim 84 wherein said second ligand-binding polypeptide is (a) an antibody or antigen-binding fragment thereof, (b) a receptor, (c) a lectin, (d) a cell adhesion molecule or (e) an extracellular matrix protein or (f) a synthetic peptide.

93. The method of claim 84 wherein
(a) said first ligand-binding polypeptide is protein A, protein G, or recombinant protein A/G; and
(b) said second ligand binding polypeptide is an antibody or antigen-binding fragment thereof.

94. The method of claim 84 wherein
(a) said first ligand-binding polypeptide is avidin or streptavidin; and
(b) said second ligand binding polypeptide is a biotinylated antibody.

95. The method of any of claim 80 wherein said first ligand-binding polypeptide is a anti-CD34 monoclonal antibody.

96. The method of claim 91 wherein said first ligand-binding polypeptide is a anti-CD34 monoclonal antibody.

97. The method of claim 89, wherein said extracellular matrix polypeptide is selected from the group consisting of collagen, laminin, fibronectin and thrombospondin 1, vitronectin, elastin, tenascin, aggrecan, agrin, bone sialoprotein, cartilage matrix protein, fibrinogen, fibrin, fibulin, a mucin, entactin, osteopontin, plasminogen, restrictin, serglycin, SPARC/osteonectin, versican, von Willebrand Factor, a cadherin, a connexin, and a selectin.

98. The method of claim 92, wherein said extracellular matrix polypeptide is selected from the group consisting of a collagen, laminin, fibronectin and thrombospondin 1, vitronectin, elastin, tenascin, aggrecan, agrin, bone sialoprotein, cartilage matrix protein, fibrinogen, fibrin, fibulin, a mucin, entactin, osteopontin, plasminogen, restrictin, serglycin, SPARC/osteonectin, versican, von Willebrand Factor, a cadherin, a connexin, and a selectin.
